(19) United States
(12) Patent Application Publication
(10) Pub. No.: US 2025/0208114 A1
(43) Pub. Date: Jun. 26, 2025

(54) HIGH SENSITIVITY AND SELECTIVITY VERTICALLY-ORIENTED SILICON NANOWIRE ARRAY-BASED BIOELECTRONIC SENSOR PLATFORM

(71) Applicant: University of Iowa Research Foundation, Iowa City, IA (US)

(72) Inventors: Fatima Toor, Coralville, IA (US); Aliasger K. Salem, Iowa City, IA (US)

(73) Assignee: University of Iowa Research Foundation, Iowa City, IA (US)

(21) Appl. No.: 18/848,689

(22) PCT Filed: Mar. 22, 2023
(86) PCT No.: PCT/US2023/064816

§ 371 Date: Sep. 19, 2024

Related U.S. Application Data
(60) Provisional application No. 63/348,103, filed on Jun. 2, 2022.
(60) Provisional application No. 63/322,559, filed on Mar. 22, 2022.

(51) Int. Cl. G01N 33/487; H10D 62/10

(52) U.S. Cl. CPC G01N 33/48785; G01N 33/48707; H10D 62/119; G01N 2333/165; G01N 2333/575

(57) ABSTRACT

A vertically-oriented silicon nanowire-array based bioelectronic sensor platform is provided which includes a vertically-oriented silicon nanowire-array bioelectronic sensor including a silicon substrate doped to function as an electrically active p-n junction diode with a p-doped base and an n+-doped emitter and having a vertically-oriented silicon nanowire-array at the n+-doped emitter, a conductive contact positioned at the silicon substrate and in electrical connection with the vertically-oriented silicon nanowire-array, a dielectric stack overlaying the conductive contact, and a back contact associated with a back surface of the silicon substrate.

### RELATED APPLICATIONS

This application claims priority to U.S. provisional patent application No. 63/322,559, filed Mar. 22, 2022, and entitled “HIGH SENSITIVITY AND SELECTIVITY VERTICALLY-ORIENTED SILICON NANOWIRE ARRAY-BASED BIOELECTRONIC SENSOR PLATFORM”. This application further claims priority to U.S. provisional patent application No. 63/348,103, filed Jun. 2, 2022, and entitled “HIGH SENSITIVITY AND SELECTIVITY VERTICALLY-ORIENTED SILICON NANOWIRE ARRAY-BASED BIOELECTRONIC SENSOR PLATFORM” both of which are hereby incorporated by reference in their entireties.

### FIELD OF THE INVENTION

The present invention relates to bioelectronic sensors or biosensors. More specially, but not exclusively, the present invention relates to a vertically-oriented silicon nanowire array-based bioelectronic sensor platform suitable for uses including, without limitation, detection of analytes such as antigens, ctDNA mutations, Coronavirus and respiratory virus spike proteins, and water contaminants.

### BACKGROUND OF THE ART

Various biosensors have been proposed. These include conventional Si nanowire (NW) biosensors based on complex field effect transistor (FET) device design. Yet problems remain with such designs including complexity of design. In addition, sensing involves measuring very small (e.g. 10⁻¹³ A to 10⁻⁷ A) currents which adds to the cost and complexity as expensive current preamplifiers are needed.

In addition, some SiNW based biosensing processes rely upon light based measurements to track changes in diode current such as those disclosed in US20170052182A1, hereby incorporated by reference in its entirety. However, light based measurements can cause unnecessary noise in the measurements due to a light source's drift overtime, unexpected shadowing due to dust, electrical probes etc.

Furthermore, there are problems with functionalization of biosensors as exposure to liquids during the functionalization process may have adverse effects on contacts of the biosensor.

Therefore, what is needed are new and improved biosensors.

### SUMMARY

Therefore, it is a primary object, feature, or advantage of the present disclosure to improve over the state of the art.

It is another object, feature, or advantage to provide a biosensor which provides considerable electrical current amplification.

Yet another object, feature, or advantage is to provide a biosensor which allows for high sensitivity without requiring expensive hardware.

Another object, feature, or advantage is to provide a biosensor with a high signal-to-noise ratio (SNR).

Yet another object, feature, or advantage is to provide a biosensor which does not require exposure to light.

It is a further object, feature, or advantage of the present disclosure to provide a silicon nanowire biosensor with low manufacturing costs and portable drive electronics.

It is a still further object, feature, or advantage to provide a scalable bioelectronic sensor platform which converts the binding of target biomolecules to electrical signals in a rapid and compact form factor.

A further object, feature, or advantage is to enable point-of-care testing of analytes such as viruses or spike proteins by minimally trained individuals.

Another object, feature, or advantage is to provide a biosensor based on a simpler manufacturing process than silicon nanowire field effect transistors thereby enabling scalability and cost-effectiveness

Yet another object, feature, or advantage is to provide a biosensor with lower noise than light-based biosensors thereby enhancing portability of the biosensor platform.

A further object, feature, or advantage is to provide a biosensor capable of high specificity detection.

A still further object, feature, or advantage is to provide a biosensor which provides for multiplexed detection relative rapid antigen tests including for coronaviruses.

Another object, feature, or advantage is to provide for a bioelectronic sensor platform which is easy to use thereby reducing the need for trained professionals to run the tests such as is needed for RT-PCR and NGS analyses.

Yet another object, feature, or advantage is to provide a biosensor which may be manufactured with the scalable and low-cost metal-assisted chemical etching (MACE) process.

One or more of these and/or other objects, features, or advantages will become apparent from the specification and claims that follow. No single embodiment need achieve each and every object, feature, or advantage as different embodiments may have different objects, features, or advantages. Therefore, the present disclosure is not to be limited to or by any object, feature, or advantage set forth herein.

According to one aspect, a vertically-oriented silicon nanowire-array based bioelectronic sensor platform is provided which includes a vertically-oriented silicon nanowire-array bioelectronic sensor including a silicon substrate doped to function as an electrically active p-n junction diode with a p-doped base and an n+-doped emitter and having a vertically-oriented silicon nanowire-array at the n+-doped emitter, a conductive contact positioned at the silicon substrate and in electrical connection with the vertically-oriented silicon nanowire-array, a dielectric stack overlaying the conductive contact, and a back contact at a back surface of the silicon substrate. The platform may further include a housing comprising a top portion and a bottom portion and configured such that wherein the top portion has an opening aligned with a sensing area comprising the vertically-oriented silicon nanowire-array of the vertically-oriented silicon nanowire-array bioelectronic sensor. The platform may further include a first conductor electrically connected to the conductive contact and a second conductor electrically connected to the back contact to provide first and second terminals for the vertically-oriented silicon nanowire-array bioelectronic sensor. The first conductor and the second conductor may both be formed of conductive tape such as copper tape. The platform may further include a layer of insulative tape between the vertically-oriented silicon nanowire-array bioelectronic sensor and the top portion of the housing. The platform may further include a current sensor electrically connected to the vertically-oriented silicon nanowire-based bioelectronic sensor. The platform may further include a processor electrically connected to the current sensor. The platform may further include a user interface device operatively connected to the processor. The user interface device may be a touchscreen display. The bioelectronic sensor may be functionalized for detection of analytes selected from a set consisting of cancer cell antigens, ctDNA mutations, coronavirus spike protein, and a hormone. The vertically-oriented silicon nanowire-array may include etched vertical silicon nanowires of at least about 350 nm in length and at least about 10¹⁰ per 1 cm² in density The platform may include a first sensor functionalized for a first analyte and a second sensor functionalized for a second analyte, the first analyte different from the second analyte. The bioelectronic sensor may be fabricated using a metal-assisted chemical etching (MACE) process. The vertically-oriented silicon nanowire array-based bioelectronic sensor may have n+-doping in a range of 7×10¹⁷ to 1×10¹⁹ cm⁻³.

According to another aspect, a vertically-oriented silicon nanowire array-based bioelectronic sensor platform includes a first vertical silicon nanowire array-based biosensor, a second vertical silicon nanowire array-based biosensor in parallel with the first vertical silicon nanowire array-based biosensor, and a current sensor electrically connected to the first vertical silicon nanowire array-based biosensor and the second vertical silicon nanowire array-based biosensor to provide for multiplexed detection of a first analyte with the first vertical silicon nanowire-array based biosensor and a second analyte with the second vertical silicon nanowire array-based biosensor. Each of the first vertical nanowire array-based biosensor and the second vertical silicon nanowire array-based biosensor comprises a silicon substrate doped to function as an electrically active p-n junction diode with a p-doped base and an n+-doped emitter and having a vertically-oriented silicon nanowire-array at the n+-doped emitter, a conductive contact positioned at the silicon substrate and in electrical connection with the vertically-oriented silicon nanowire-array, a dielectric stack overlaying the conductive contact, and a back contact at the back surface of the silicon substrate. The first vertical silicon nanowire array-based biosensor may be functionalized for detecting the first analyte and the second vertical silicon nanowire array-based biosensor is functionalized for detecting the second analyte.

According to another aspect, a method includes fabricating a vertically-oriented silicon nanowire array-based bioelectronic sensor comprising a silicon substrate doped to function as an electrically active p-n junction diode with a p-doped base and an n+-doped emitter and having a vertically-oriented silicon nanowire-array at the n+-doped emitter, a conductive contact positioned at the silicon substrate and in electrical connection with the vertically-oriented silicon nanowire-array, a dielectric stack overlaying the conductive contact, and a back contact at a back surface of the silicon substrate. The method further includes biofunctionalizing the vertically-oriented silicon nanowire array-based bioelectronic sensor. The method may further include calibrating I-V curves for the vertically-oriented silicon nanowire array-based bioelectronic sensor.

According to another aspect, silicon nanowires are next-generation high performance biosensor materials compatible with multiple types of biomolecules. Bioelectronic sensors, which outputs electrical signals for biological detection, have unique advantages in miniaturization, fast response, and portability. Despite that these nanomaterials have demonstrated high performance, complex fabrication methods that are not compatible with industrial production are usually implemented. The present disclosure addresses the development, fabrication, and testing of a rapid and cost-effective silicon nanowire biosensor that may be less than one inch in width and suited for industrial mass production. The silicon nanowires are fabricated using a silver-assisted chemical etching which has compatibility with mass production, tunable etch rate, and high consistency. The nanowire sensor is then fabricated using a series of nanofabrication instruments that are commonly used for semiconductor processing. The fabrication process is developed and modified to be suited for biosensing applications, and the scanning electron microscopy demonstrates that the fabricated sensor has etched vertical silicon nanowire arrays of around 350 nm in length and 10¹⁰ per 1 cm² in density.

The fabricated vertically-oriented silicon nanowire array-based sensor includes a p-n diode. The present disclosure describes implementation of the diode type nanowire biosensors. The present disclosure describes functionalizing the biosensors the testing of different types of analytes including (i) two cancer cell antigens, (ii) ctDNA mutations, (iii) Coronavirus spike protein, and (iv) estrogenic compounds that are water quality contaminants. The results show that the developed sensors have high sensitivity and specificity.

The biosensor has already demonstrated detection of clinically relevant concentrations of the target entities for high reliability diagnosis and monitoring of disease. This technology offers the potential to complement conventional biosensor systems in numerous applications including applications in portable and rapid responding biosensing.

### BRIEF DESCRIPTION OF THE DRAWINGS

FIG. 1 is a pictorial overview of a vSiNW biosensor such as may be used for the encapsulation for SARS-CoV-2 detection. (a) Schematics of the detection mechanism of the vSiNW biosensor. Spike proteins exist on the surface of SARS-CoV-2, and the spike can specifically bind to the ACE-2 protein that is immobilized on the surface of vSiNWs. Negative electrostatic surface potential is induced by the spike protein after binding, which enhances the surface recombination effect if the vSiNWs are n⁺-doped. (b) Photo of a fabricated vSiNW biosensor. (c) Titled top and cross-sectional view schematic diagrams of the vSiNW biosensor. (d) Structural schematic of the ABS encapsulation of the vSiNW sensor, the assembly starts from the bottom. (e) I(V) testing system of the vSiNW biosensor with the sensor encapsulation, an external source meter, the analyte on the sensing area, and an ABS cover on top.

FIG. 2 is a view of one example of a vSiNW biosensor.

FIG. 3 is a further view of an encapsulation of the vSiNW sensor as shown in panel (d) of FIG. 1.

FIG. 4 illustrates the vSiNW biosensor with sensor encapsulation as shown in FIG. 1 panel (e).

FIG. 5 illustrates one example of a sensor platform including the vSiNW sensor.

FIG. 6 illustrates one example of a method of manufacturing the vSiNW sensor. (a) Process flow of one-step MACE of Si NWs. An immersion in DI water is included after each step. (b) Schematic process flow of the fabrication of the vSiNW biosensor. (c) Cross-sectional and (d) tilted top-down SEM images of vSiNWs right after etching. (e) Cross-sectional and (f) tilted top-down SEM images of vSiNWs after fabrication. The difference in morphologies of vSiNWs is attributed to the oxidation damage during high-temperature doping.

FIG. 7 illustrates design and validation of the ACE-2-Fc and Spike-Fc soluble probes. (a) Composition of the ACE-2-Fc chimeric protein. The extracellular domain of human ACE-2 was fused to the Fc region of human IgG1. A thrombin cleavage site was introduced to allow removal of the Fc region. (b) Analysis of the purity and size of the ACE-2-Fc and Spike-Fc probes by polyacrylamide gel electrophoreses, followed by silver staining. (c) Detection of the ACE-2-Fc probe by cell-based ELISA. HOS cells were transfected to express on their surface the SARS-CoV-2 spike protein. As controls, some samples were transfected with an empty (mock) vector. Binding of ACE-2-Fc to the cells was detected using an HRP-conjugated goat anti-human IgG preparation. Significance of the differences between binding to cells that express spike and mock-transfected cells was calculated using an unpaired t-test: *, P<0.05; **; P≤0.005; ***, P≤0.0005. (d) Composition of the soluble Spike-Fc probe. To generate a probe that contains both S1 and S2 subunits, the furin cleavage site was disrupted by substitution of the Arg-Arg-Ala-Arg motif to Ser-Ser-Ala-Ser. (e) Measurements of Spike-Fc binding to HOS cells transfected by a plasmid that expresses the ACE-2 protein or a negative control (mock) plasmid.

FIG. 8 illustrates a study of the three-diode model for the vSiNW biosensor. (a) Equivalent circuit diagram of the proposed three-diode model for the vSiNW biosensor. (b) Example schematic diagram of the possible location of each component of the circuit in (a). Variations of simulated I_{D}-V_{D} curve for different values of I_{0H} and R_{H}=100Ω (c) and 1000Ω (d). Other parameters: I₀₁=10⁻¹¹ A, I₀₂=1×10⁻⁷ A, n₁=1, n₂=2, n_{H}=2.5, Rₛ=0Ω, Rₛₕ→∞.

FIG. 9 illustrates calibration of I(V) curves. Measured I(V) curves of an AS (a) and a BB sensor (b). Calibrated I(V) (I_{D}-V_{D}) curves of the AS (c) and the BB sensor (d).

FIG. 10 illustrates specificity and concentration response results of the vSiNW biosensor for spike protein detection. (a) Statistics of ΔI_{D} % at V_{D}=100 m V for the positive test AS and three negative control tests AB, BS, and BB. (b) ΔI_{D} % at V_{D}=100 mV of the ACE-2-functionalized vSiNW biosensors at different concentrations of spike testing solutions, where the black circle represents the AID % for 0 ng/ml. (*:p<0.05, **:p<0.01, unpaired t-test between AS and each control group).

FIG. 11. Testing of the sensor device for wet measurement. (a) IV characteristic of a dummy sensor for testing with 150 μl of 0.1× PBS on the sensing area. (b) The current change ΔI and (c) ΔI % of the sensor after each time of PBS wash by removing and adding 150 μl of the solution. The 5th wash also involves shaking the sensor and moving the probes. The area between two dashed lines in (c) designates the 2% change range.

FIG. 12 (a) The dark IV (solid) and GV (dashed) curves of a fabricated sensor. (b) The InI and n_{eff} curves as functions of V_{D} for the sample in (a). The huge hump in n_{eff} indicates the localized enhanced-recombination mechanism in addition to those in the two-diode model.

FIG. 13 (a) Schematic diagram of the proposed water-gate experiment. PR=photoresist, V_{G}=gate voltage, I_{G}=gate leakage current, V_{S}=sensor voltage, I_{S}=sensor current. (b) Photo of the SiNW sensor with the photoresist. (c) Results of the sensor I_{S}−V_{S} curves under negative VG from 0 V to −6 V with a step size of 1 V. (d) Rough estimation of the I_{0H} given calculated resistances and estimated parameters of saturation currents and R_{H}. I₀₁=10⁻¹¹ A, I₀₂=2×10⁻⁷ A, Rₛ=10Ω, Rₛₕ=800,000Ω, R_{H}=5,000Ω. (e) I_{S}−V_{S} and (f) ΔI_{S} %−V_{S} curves of the sensor at V_{G}=10 V and V_{G}=−10 V.

FIG. 14 illustrates one example of a platform for biosensing. The prototype of the portable functionalized vSiNW sensor. (A) Electrical circuit schematic of the system. (B) vSiNW biosensor cartridge. (C) Touch screen at front of the device. (D) Current versus time measurement collected from the sensor for one min (one scan every two seconds). (E) Backside of the portable device, showing all system components.

FIG. 15 provides a visual summary of the nanofabrication process steps for the Si NW biosensor. The front emitter and back surface field (BSF) of the p-n junction are formed concurrently in a rapid thermal annealing furnace. Edge isolation is performed using reactive ion etching (RIE). The top metal contact pattern is exposed using photolithography, followed by a HF clean and then a titanium (Ti) and silver (Ag) deposition using an electron-beam evaporator. The silicon nitride (SiNx)/silicon dioxide (SiOx) stacks are deposited using a sputterer.

FIG. 16 provides a visual summary of the biofunctionalization steps of Si NW biosensor utilized to detect hormones. The nanofabricated Si NW biosensor is functionalized using (3-aminopropyl) triethoxysilane (APTES), followed by glutaraldehyde. This bifunctional linker allows for the immobilization of the estrogen receptor-alpha (ER-α) on the Si NW surface. The ER-functionalized surface was then passivated with ethanolamine to minimize unspecific binding. Next the target hormone species was introduced on the Si NW biosensor surface for detection.

FIG. 17 illustrates stereochemical structures of a) testosterone, b) estrone (E1), and c) estradiol (E2).

FIG. 18 illustrates JV curves before functionalization (solid line) and after using a) amino PEG (dashed line), showing the strong degradation that occurred in the biosensor versus b) ethanolamine (dashed line), where the degradation is significantly less.

FIG. 19 provides fluorescence data taken using a confocal microscope showing a) all the receptors clustered around the edge of the biosensor when no shaking was used, and b) significant improvement of uniformity of the receptors on the surface of the biosensor due to the use of a shaking table. The insets demonstrating the distribution of the receptors on the surface.

FIG. 20 illustrates a) The relationship between current density (ΔJ) and E2 concentration for biosensors at 500 Ω/sq (black dots) and 1200 Ω/sq (blue squares) showing that ΔJ increases at different rates for the two different Rₛₕₑₑₜ values. The semi-logarithmic regression fits and equations at 500 Ω/sq (black solid line) and 1200 Ω/sq (blue solid line) are also shown. b) Relationship between ΔJ and Rₛₕₑₑₜ for biosensors exposed to 10 ng/ml of E2 (black dots) and 100 ng/ml of E2 (blue squares) indicates that for higher Rₛₕₑₑₜ values, ΔJ is higher for the same E2 concentration. The linear regression fits and equations of 10 ng/mL of E2 (black solid line) and 100 ng/ml of E2 (blue solid line) are also shown.

FIG. 21 provides schematics illustrating the self-assembly processes for the immobilization of capture DNA and hybridization of the target DNA on the vSiNWs surface. (1) The Si NW presenting a native oxide on its surface was functionalized with APTES. (2) Glutaraldehyde was linked to the amino groups. (3) Terminal 3′-amino groups of the capture DNA strands were chemically bound to the aldehyde groups. (4) Complementary Alexa 488 fluortagged target DNA was hybridized to the capture DNA on the vSiNWs surface.

FIG. 22 shows a mean fluorescence comparison of the etched vSiNWs relative to unetched planar Si analyzed using imageJ, values expressed in mean±SD, significant differences were assessed by two-way ANOVA with Tukey's test. Inset shows the fluorescence images of the NWs and planar surfaces functionalized with fluorotagged target BRAF V600E DNA. Scale bars=200 μm.

FIG. 23 illustrates one example of a work flow for self-assembly of capture DNA on the SiNWs surface.

FIG. 24 illustrates an example of a workflow for use in immobilization of DNA on SiNW.

### DETAILED DESCRIPTION

The biosensors and platform described herein are suitable for a number of uses including, without limitation, detection of analytes such as antigens, ctDNA mutations, Coronavirus and respiratory virus spike proteins, and water contaminants. The present disclosure is not to be limited by or to specific analytes. This description begins with a brief overview of the basic structure of the biosensor and example of a manufacturing process. Next, the biosensor is described with respect to COVID-19 spike protein testing. Then addition examples are provided for different analytes.

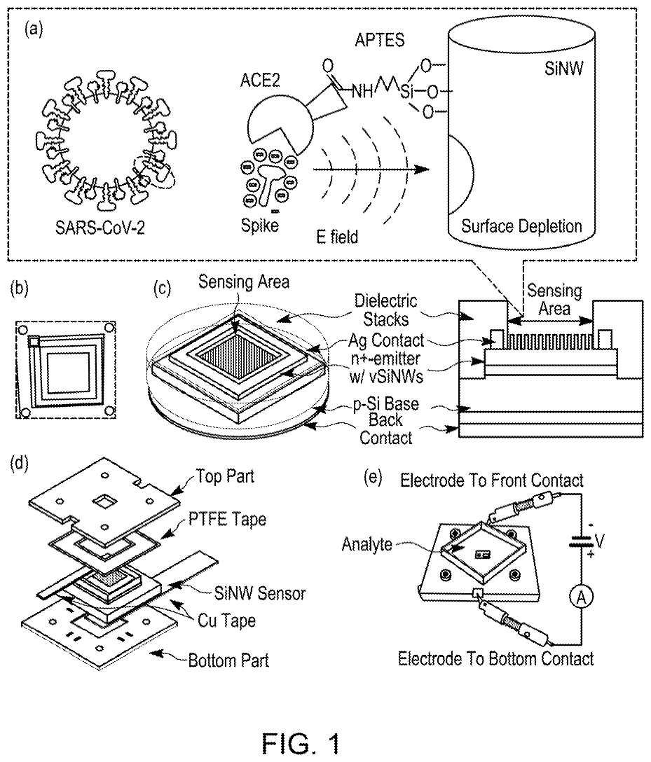

FIG. 1 is a pictorial overview of a vSiNW biosensor such as may be used for the encapsulation for SARS-CoV-2 detection (as shown) or detection and/or quantification of other analytes. Although shown in the context of being functionalizes for SARS-CoV-2 spike protein detection, the vSiNW biosensor may be adapted to detection of different types of analytes.

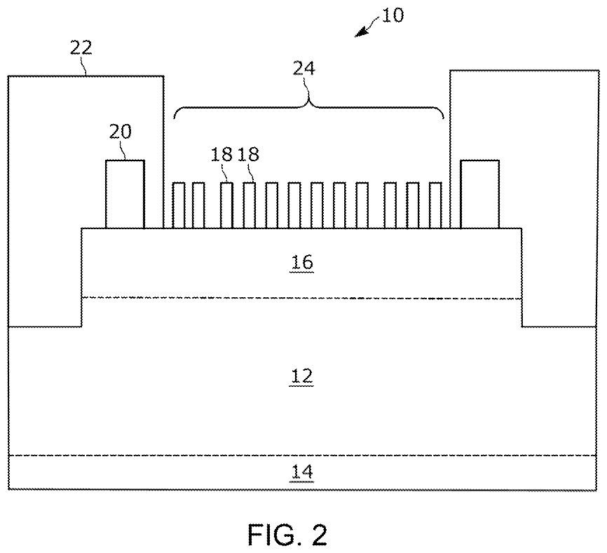

FIG. 2 is a view of one example of a vSiNW biosensor. In FIG. 2, a vSiNW biosensor 10 is shown. A substrate is shown with a p-Si base region 12, and an n+-doped emitter region 16 which includes an array of vertical Silicon nanowires 18. A contact 20 is shown which may comprise silver. Overlaying the contact 20 is a dielectric stack which serves as a moisture barrier. A sensing area 24 where an analyte may be brought in contact with the array of vertical Silicon nanowires 18. Below the p-Si base region 12 is a back contact 14.

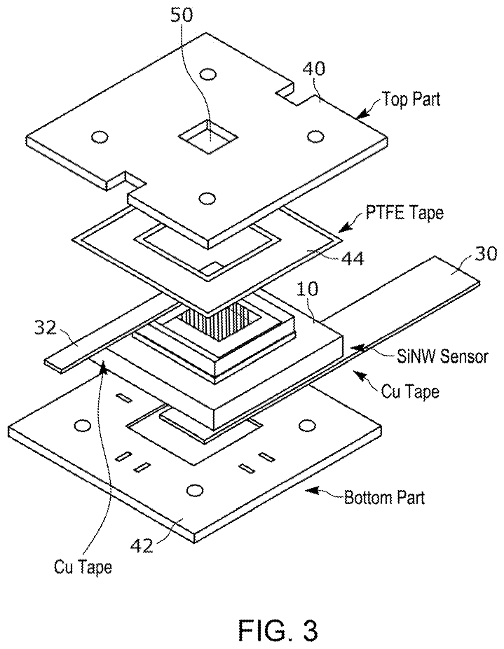

FIG. 3 is a further view of an encapsulation of the vSiNW sensor as shown in panel (d) of FIG. 1. The encapsulation or housing for the vSiNW biosensor 10 includes a top portion 40 and an opposite bottom portion 42 such that the vSiNW biosensor 10 is positioned therebetween. Conductive tape such as copper tape 40 may be electrically connected to a back contact of the vSiNW biosensor 10. Similarly, conductive tape such as copper tape 32 may be electrically connected to the contact which may be formed of silver. A layer of polytetrafluoroethylene (PTFE) tape 44 may be placed between the sensor and the top portion to help position and seal the top portion of the encapsulation or housing in place. There is an opening 50 in the top portion 40 which allows for access to the sensing area of the vSiNW biosensor.

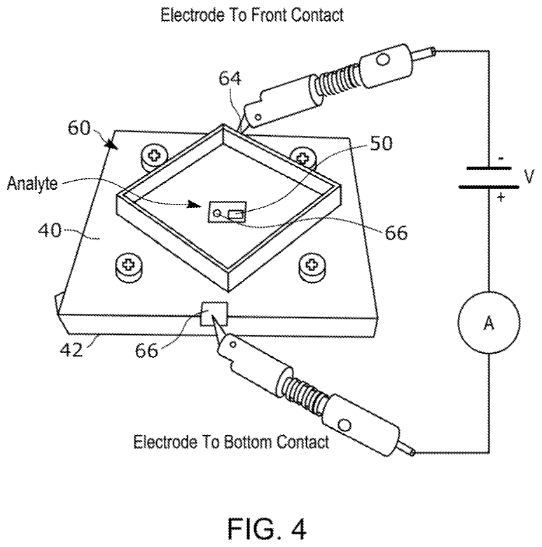

FIG. 4 illustrates the vSiNW biosensor with sensor encapsulation as shown in FIG. 1 panel (e). There is a housing 60 having a top portion 40 and a bottom portion 62. A front contact 64 and a bottom contact 66 are accessible. An analyte 46 is positioned within the opening 50 of the top portion 40 to come in contact with the sensing area of the vSiNW biosensor.

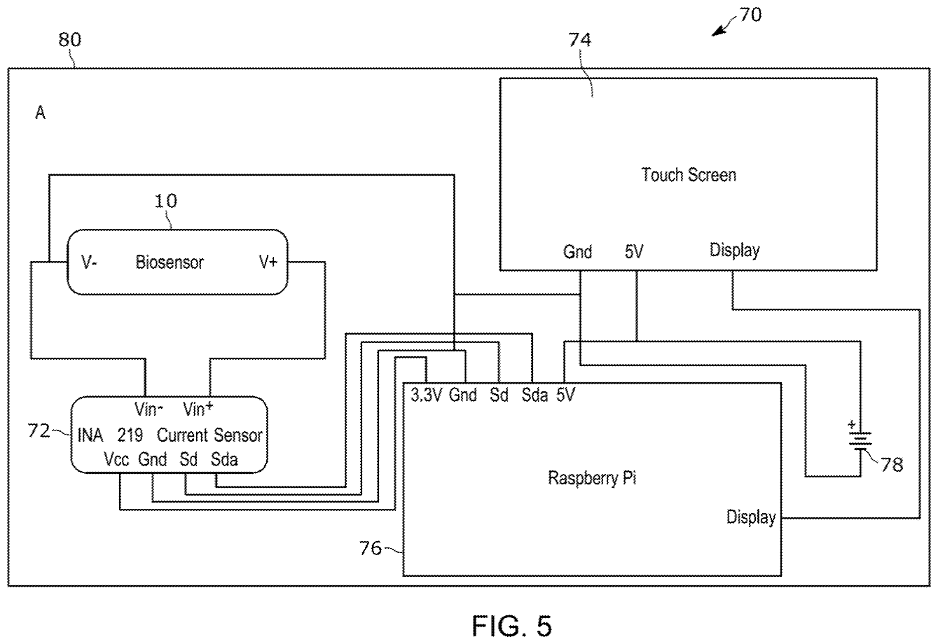

FIG. 5 illustrates one example of a sensor platform including the vSiNW sensor. The platform 70 includes one or more vSiNW biosensors 10. Where multiple vSiNW biosensors are present, they may be placed in parallel. The vSiNW biosensor 10 is electrically connected to a current sensor 72. The current sensor 72 is electrically connected to a microcontroller 76 such as a Raspberry Pi. A user interface such as a touch screen 74 is also electrically connected to the microcontroller 76. A housing 80 is also shown which may be a portable housing and may contain its own power source 78 such as a battery.

The sensor platform may be in the form of a prototype portable biosensor platform that includes a Raspberry Pi microcontroller, an 8-channel ADS7828 high precision current sensor, and a touchscreen which is powered by a 5V battery. The vSiNW biosensor's front (negative) and back (positive) electrical contacts are connected to the ADS7828 current sensor. The Raspberry Pi runs a Python script-based Graphical User Interface (GUI) that is used to input sensor testing parameters and measure biosensor current changes and store them on an SD card. It is contemplated that a wifi enabled interface of the Raspberry Pi may be used for real-time data transfer from the biosensor to a secure (HIPAA compliant) server, and then to an easy to interpret GUI integrated in a mobile device application. The implementation of the mobile app may be used to transmit test results to relevant locations. In some applications, this may include clinics and government agencies.

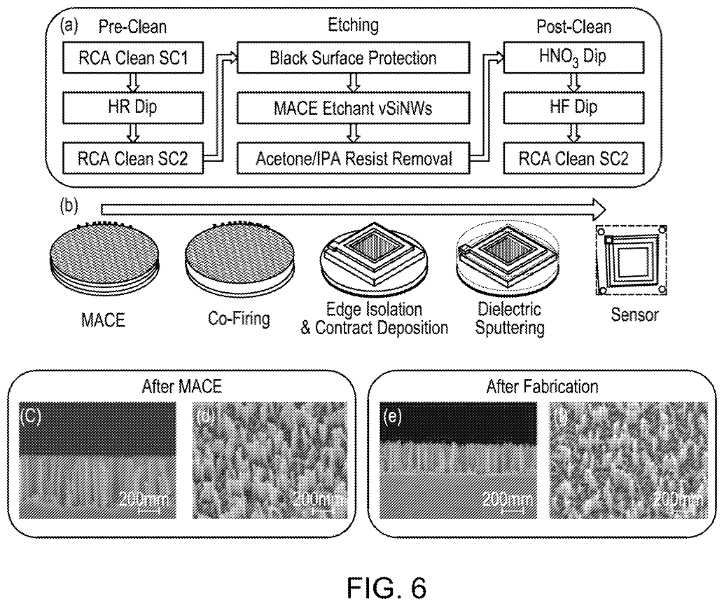

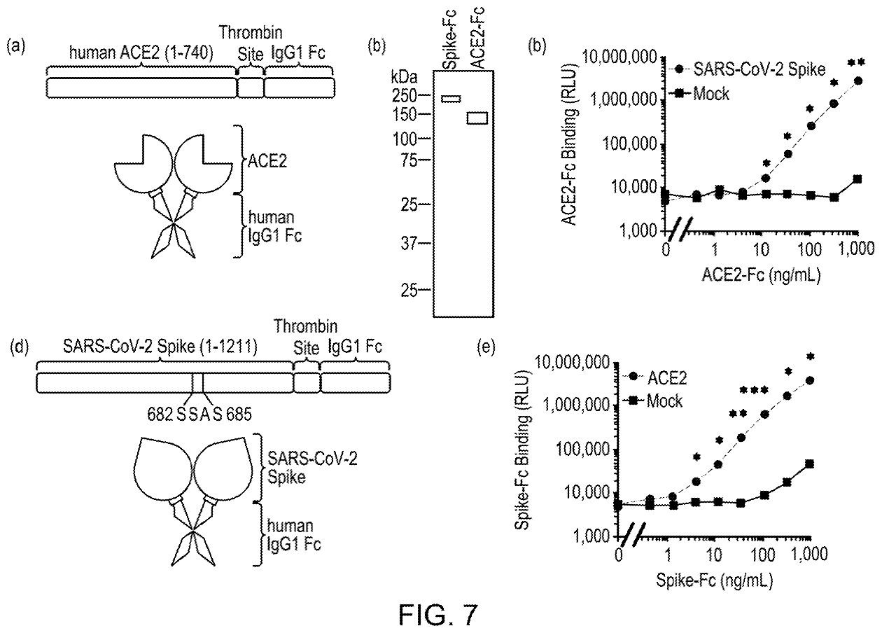

FIG. 6 provides an overview of the process of the fabrication of vSiNW biosensors. Si wafers ((100) oriented, Czochralski (CZ) processed, p-type boron doped, resistivity=1-10 Ω, 270 μm thick, 2 in diameter) were purchased from University Wafer. All the organic chemical solvents including ethanol, acetone, and isopropyl alcohol (IPA) were LC/MS grade.

A one-step MACE process was applied as shown in panel (a) of FIG. 6. Si wafer was first cleaned using the standard Radio Corporation of America (RCA) protocol. The RCA standard clean (SC) 1 solution comprised ammonium hydroxide (NH₄OH), H₂O₂, and H₂O in a volume ratio of 1:1:5, and the SC 2 solution had hydrogen chloride (HCl), H₂O₂, and H₂O in the same ratio. Both solutions were heated up to 80° C. A 10% hydrofluoric acid (HF) solution was used between two clean steps to remove the oxide layer. After clean, the backside of the wafer was carefully painted with photoresist to avoid etching. The MACE etchant composed of 5 M HF and 20 mM AgNO₃, and a stirrer was slowly rotating on the bottom for quicker replenishment of consumed ions on the etching sites. After etching and washing off the backside photoresist by acetone and IPA sequentially, Ag dendrites grown during etching were removed in a nitric acid (HNO₃) solution with 68% concentration, followed by the HF dip to remove SiO₂. Finally, the RCA SC 2 clean was conducted again to ensure thorough removal of Ag atoms, which would however also etch a thin layer of Si from the NWs. The wafer was immersed in deionized (DI) water after each step.

The biosensor was then fabricated according to the process shown in FIG. 6, panel (B). Scanning electron spectroscopy (SEM) images of vSiNWs after etching before doping are shown in FIG. 6, panels c and d. After wafer clean and SiNW etching for 5 min, the annealing and doping of both the front and the back sides were completed in a one-step co-firing process. The Al paste was first screen-printed on the back side of the sensor substrate. Then another planar Si substrate, denoted by the source substrate, was spin-coated ammonium dihydrogen phosphate (NH₄H₂PO₄, ADP) on the front side and dried on a hot plate at 100° C., and the concentration of ADP was tuned to make sure that the sheet resistance (Ra) of the emitter was consistently around 1000 Ω·□⁻¹. Then both substrates were inserted in a rapid thermal annealer (RTA), front side to front side, with a 0.5 mm spacing, and the chamber of the RTA was heated at 950° C. for 10 min. During the process, Al back surface field (BSF) was formed through the mutual diffusion of Al and Si atoms, while the phosphorous (P) dopant diffused into the source substrate through direct diffusion and to the sensor surface through proximity diffusion. After co-firing, the length of SiNWs decreased from around 450 nm to around 350 nm, and the NW thinning and separating effects were also demonstrated in the SEM images in FIG. 6, panels e and f. After doping, the front surface had a coating of n+-doped silicon oxide named phosphosilicate glass (PSG), which was then removed by an HF immersion. A 200 nm thick Ag layer was deposit on the Al BSF for better conductivity and protection of Al from alkaline developers for the following photolithography. An 11.5 mm by 11.5 mm sensor active area was patterned and covered by photoresist on the HF cleaned surface, followed by an edge isolation that etched off SiNWs and more than 500 nm conductive n-type emitter outside the active area through RIE. Then the patterned Ag busbar was deposit on the SiNWs through photolithographic lift-off and e-beam deposition. Finally, the front surface except for a sensing area of 8.6 mm by 8.6 mm and a contact pad of 1 mm by 1 mm was coated with dielectric stacks of SiOx/SiNx/SiOx/SiNx that were 50 nm/100 nm 50 nm/100 nm thick.

However, to aid in the discussion, the biosensors will be discussed first in the context of COVID-19 testing and SARS-CoV-2 spike protein detection with high sensitivity and selectivity. However, the present invention is not to be limited to this specific application.

### Example 1—Covid-19

The COVID-19 pandemic has caused tremendous damage to the world. In order to quickly and accurately diagnose the virus and contain the spread, there is a need for rapid, sensitive, accurate, and cost-effective SARS-CoV-2 biosensors. In this paper, we report on a novel biosensor based on angiotensin converting enzyme 2 (ACE-2)-conjugated vertically-oriented silicon nanowire (vSiNW) arrays that can detect the SARS-CoV-2 spike protein with high sensitivity and selectivity relative to negative controls. First, we demonstrate the efficacy of using ACE-2 receptor to detect the SARS-CoV-2 spike protein via a capture assay test, which confirms high specificity of ACE-2 against the mock protein, and high affinity between the spike and ACE-2. We then report on results for ACE-2-conjugated vSiNW arrays where the biosensor device architecture is based on a p-n junction transducer. We confirm via analytical modeling that the transduction mechanism of the biosensor involves induced surface charge depletion of the vSiNWs due to negative electrostatic surface potential induced by the spike protein after binding with ACE-2. This vSiNW surface charge modulation is measured via current-voltage characteristics of the functionalized biosensor. Calibrated concentration dependent electrical response of the vSiNW sensor confirms the limit-of-detection for virus spike concentration of 100 ng/ml (or 575 pM). The vSiNW sensor also exhibits highly specific response to the spike protein with respect to negative controls, offering a promising point-of-care detection method for SARS-CoV-2.

### 1. Introduction

Coronavirus disease 2019 (COVID-19) is a human infectious disease emerged in late 2019 that is caused by Severe Acute Respiratory Syndrome Coronavirus 2 (SARS-CoV-2). Based on the rapid increase in human infection, the World Health Organization has classified the COVID-19 outbreak as a pandemic. Among people without vaccination or immunity, early diagnosis and containment are critical for limiting the spread of the virus [1]. One approach to detect SARS-CoV-2 infection is the reverse transcription polymerase chain reaction (RT-PCR) using nasopharyngeal (NP) or throat swab samples [2]. It involves incubation, RNA extraction, reverse transcriptase, PCR amplification, and spectrophotometry [3]. This approach has good specificity but intermediate sensitivity, with false-negative rates ranging between 4% and 29% [4, 5]. In particular, the sensitivity and false-negative rates can aggravate five days after symptom onset [6, 7]. According to the Center for Disease Control [8], while RT-PCR based tests are highly specific and sensitive, they are costly (˜$75-$100/test) with moderate to high complexity sample processing requiring trained professional staff, and the turnaround time for results could range from several hours to 1-3 days. The considerable lag between sample collection and the time individuals are informed of the results is a major concern and can lead to preventable spread of SARS-CoV-2. RT-PCR based tests also require Clinical Laboratory Improvement Amendments (CLIA) certification which can be costly to obtain and maintain for resource constrained rural clinics [9].

Several inexpensive point-of-care (POC) antigen-detection rapid diagnostic tests (Ag-RDTs) are currently commercially available. World Health Organization (WHO) recommends minimum sensitivity of 80% and specificity of 97% for Ag-RDTs [10]. Peeling et al. reported in an extensive global study of Ag-RDTs that almost all of the single antigen tests for SARS-CoV-2 meet the WHO criteria. WHO recognizes that despite lower sensitivity than RT-PCR tests, Ag-RDTs offer the possibility of rapid, inexpensive detection of SARS-CoV-2 in individuals who have high viral loads and hence are at high risk of transmitting the infection to others. However, some key shortcomings are that Ag-RDTs are typically non-modular i.e., are designed to detect only one virus, and cannot differentiate between SARS-CoV-2 and other common respiratory viruses. Ag-RDTs tests also only provide qualitative results with limited ability to identify the stage of infection.

Alternatively, recently there has been increasing efforts in the development of angiotensin-converting enzyme 2 (ACE2) receptor-based biosensors by multiple research groups around the world [12-16]. This is because ACE2 is the main receptor used by SARS-CoV-2 for cellular entry, and infection of SARS-CoV-2 begins when the spike protein binds to ACE2 [17]. In a recent study, Ozono et al. reported the binding affinity of ACE2 with five variants having global spread and mutations in the spike protein, and noted that four out of five variants showed significantly increased binding affinity to ACE2. From these results, one can infer that variants with a higher binding affinity to ACE2 are more contagious. Accordingly, implementing ACE2 in a biosensor can be an effective strategy for screening variants with high transmissibility.

Recently Park et al. demonstrated a high sensitivity (˜165 virus copies/mL) dual-gate field-effect transistor (FET) using ACE2 as the receptor. However, the dual-gate FET architecture was rather complex to manufacture and based on non-CMOS compatible materials, such as tin oxide, titanium nitride, among others; all of which can make it challenging to scale up this biosensor architecture for global monitoring of the pandemic. The work also did not confirm selective detection of the spike with respect to negative controls. Another biosensor architecture was reported by Pinals et al. based on ACE2-single-walled carbon nanotube (SWCNT) optical sensing. The team demonstrated that the ACE2-SWCNT nanosensors exhibit a 73% fluorescence turn-on response within 5 seconds of exposure to 35 mg/L SARS-CoV-2 virus-like particles. SWCNTs are expensive and spectroscopic measurements needed for the optical characterization require bulky equipment that renders this sensor architecture unsuitable for inexpensive portable biosensors.

In this disclosure, we present results on a scalable bioelectronic sensor platform, which converts the binding of target biomolecules to electrical signals in a rapid and compact formfactor, enabling POC testing of viruses, such as COVID-19, by minimally trained individuals qualifying it for eventual CLIA waiver certification. Our biosensor architecture is based on silicon nanowires (SiNWs) that various teams [19, 20] including ours have demonstrated to have higher sensitivity than planar Si biosensors because of the high surface-to-volume ratio of the SiNWs. However, not all SiNWs-based biosensors are suited for COVID-19 detection, because their manufacturing steps are not complementary metal oxide semiconductor (CMOS) compatible while also being low-yield, slow, and require high-cost equipment [22-25]. To address these challenges of SiNWs-based biosensors, we employ the metal-assisted chemical etching (MACE) process [26, 27] to fabricate large area arrays of vertically-oriented SiNWs (vSiNWs) for our biosensor. The MACE process is CMOS-compatible and does not require any vacuum-based equipment and can be scaled up cost-effectively. FIG. 6 shows the scanning electron microscope images of the resulting vSiNWs from the MACE process. After the vSiNW MACE etching, the Si substrate is turned into an electrically active p-n junction diode via a number of nanofabrication steps shown in FIG. 6. Our simple device architecture allows us to electrically probe millions of NWs concurrently via a top and bottom contact scheme (FIG. 1(c)), and since each NW acts as an anchor for the target molecule, the sensor architecture results in highly sensitive detection of the biomolecules.

We functionalized the vSiNWs-based biosensors for SARS-CoV-2 detection by first treating the sensor surface with (3-Aminopropyl) triethoxysilane (APTES) and then immobilizing human angiotensin converting enzyme 2 (ACE-2) on APTES. Since ACE-2 interacts with the spike protein of SARS-CoV-2 with high affinity [28], we decided to utilize this interaction to detect presence of the virus. The spike protein is on the surface of SARS-CoV-2 molecule, and the specific binding of spike and ACE-2 proteins is found to be critical to the immobilization of SARS-CoV-2 during the infection of human cells [29]. A similar biochemical process was simulated on the surface of vSiNWs when the ACE-2 functionalized sensor was immersed in the solution of spike protein. We expect the polar spike protein to induce an electrostatic surface potential that can modulate the carrier density of vSiNWs substantially [30], as shown in FIG. 1(a). The detection of the coronavirus was thereby enabled by the conversion of this charge density modulation in the n-doped vSiNWs, into an electrical signal. Relative to RT-PCR, advantages of the vSiNW sensor platform are manifold, including: (i) results are obtained within one hour, allowing rapid containment of infected individuals, (ii) due to the low expense of fabricating Si devices, the cost of each sensor is expected to be $10 to $15 (at scale production of 100,000 sensors/year), (iii) the sensor is small and portable, and can be used in a wide range of public settings for on-the-spot results, and (iv) it does not require scientifically qualified personnel for operation reducing labor costs included for virus testing.

### 2. Material and Methods

### 2.1.1. Design of Probes to Detect SARS-CoV-2 Spike Protein

The ACE-2 protein is bound to the surface of cells and serves as an entry receptor for SARS-CoV-2 [31]. To this end, we first generated a soluble form of ACE-2, by introducing a stop codon at the C-terminal position of the extracellular domain (at amino acid position 741). This domain was linked to the Fc region of human IgG1 (FIG. 7(a)), which allows purification of the chimeric protein using Protein A-coated beads and renders the protein dimeric. A thrombin cleavage site was introduced between the ACE-2 and the Fc segment for removal of the latter after purification [32]. Molecular weight and purity of the chimeric protein was validated by silver staining of samples analyzed by polyacrylamide gel electrophoresis (FIG. 7(b)). To determine the ability of the ACE-2-Fc probe to detect spike, we used a cell-based enzyme-linked immunosorbent assay (ELISA) [33, 34]. For this purpose, we transfected human osteosarcoma (HOS) cells with a plasmid that expresses the SARS-CoV-2 spike protein. Cells were then incubated with soluble ACE-2 and its binding was detected using a horseradish peroxidase-conjugated secondary antibody and measured by luminescence. The limit of detection for binding of ACE-2-Fc to the spike-expressing cells was 12.5 ng/mL (96.2 pM) (FIG. 7(c)).

To quantify the ability of the ACE-2-activated vSiNWs to detect spike, we also generated a soluble form of the spike protein. It is composed of the ectodomain of spike (amino acids 1-1211), a Thrombin cleavage site, and the Fc region of human IgG1 (FIG. 7(d)). To maintain subunits S1 and S2 of spike associated, we abrogated the furin cleavage site at positions 682-685 by substituting the existing Arg-Arg-Ala-Arg cleavage motif to Ser-Ser-Ala-Ser. Binding efficiency of the chimeric Spike-Fc construct to ACE-2 was measured by cell-based ELISA using HOS cells transfected with ACE-2. As shown in FIG. 7(e), binding of Spike-Fc to the ACE-2-expressing cells was detected at a concentration as low as 0.4 ng/ml (2.3 pM). We note that viral loads in the upper airways during early stages of infection are ˜10⁶-10⁹ RNA copies per mL [35]. Assuming 30 spikes per virion [36], this corresponds to 0.05-50 pM of spike protein. Thus, the detection limit of the Spike-Fc probe using the cell-based ELISA system is within this range.

2.2. Preparation of Testing Solutions for the vSiNW Biosensor

For testing the vSiNW sensor, two types of proteins were prepared. Spike proteins were prepared and used for the specific binding with the ACE-2 on the functionalized sensor surface. Bovine serum albumin (BSA) was used as a disturber for the specificity test, because its structure is analogous to that of the human serum albumin (HSA) [37]. Both BSA and spike were dissolved in the phosphate-buffered saline (PBS) at a serum-like pH of 7.4, and the concentration of all PBS solutions was 0.1x giving a sufficiently high Debye length of around 2.4 nm [38]. An isoelectric point (pI) study of the spike protein showed that it has a pI of around 5, which means it applied a substantial negative potential in PBS.

2.3. vSiNW Biosensor Fabrication and Characterization Approaches

The photo and schematic of the vSiNW sensor are shown in FIGS. 1(b) and (c), respectively. For further details on the biosensor fabrication process, please refer to sections S1-S4 of the Supporting Information document. Briefly, a n⁺-p junction was formed on the front side of a lightly doped p-type Si substrate. The vSiNWs were turned into a highly-doped n⁺-emitter using ammonium dihydrogen phosphate (NH₄H₂PO₄, ADP), a phosphorous (P) dopant of Si [40]. The back side of the Si substrate was highly p-doped via an aluminum (Al) based back surface field. After biosensor fabrication, the resulting vSiNWs were ˜350 nm in length with a density of around 10¹⁰ SiNWs per cm² area (see FIG. 6). The sensing area was 6.2 mm by 6.2 mm, and the top contact was silver (Ag) based and the back contact of the sensor is Ag-coated Al. On the front side, the Ag-contact and Si area outside the sensing area was coated with sputtered dielectric materials, namely silicon dioxide (SiOx) and silicon nitride (SiNx) to minimize degradation of the Ag-contact during biosensor functionalization steps.

We designed a 3D-printed mount for testing the biosensors such that sufficient volume of the spike solution can be maintained on the front surface of the biosensor. The 3D-printed mount was made of two acrylonitrile butadiene styrene (ABS) plastic accessories and attached to the front and back sides of the vSiNW biosensor to form a cuvette that could contain a maximum of 250 μl of liquid on the sensing area, as shown in FIG. 1(d). Polytetrafluoroethylene (PTFE) tapes were inserted between the top plastic part and the vSiNW sensor for fluid leak proofing. Biosensor characterization was performed on a benchtop Keithley 2400 source meter unit (SMU) without illumination, as in FIG. 1(e). Front and back contacts of the sensor were extended by copper (Cu) tape for contacting with the probes connected to the SMU inputs and outputs. The analyte testing solution was added in the cuvette to cover the biosensor sensing area, and a plastic cover was capped on top to prevent evaporation and contamination of the solution during experiments. When the voltage source was turned on, the vSiNW biosensor operated as an n⁺-p diode, and the measured dark current I as a function of voltage V, I(V), was measured six times during each experiment. After assembly of the ABS parts with the sensor, the sensing area was washed by adding and removing 250 μl of PBS twice using a micropipette, then 150 μl of PBS was added, and the first I(V) curve was measured with the plastic cap on, denoted by IPBS (V). After that, the PBS was extracted, and 150 μl of testing solution was added, followed by an immediate measurement of the I(V) curve, denoted by I₀ₘᵢₙ(V). Then after each 20 min, an I(V) curve was measured until one hour after the addition of the testing solution, which generated three I(V) curves, I₂₀ₘᵢₙ(V), I₄₀ₘᵢₙ(V), and I₆₀ₘᵢₙ(V). The electrical response of the sensor was then determined by the normalized I(V) change before and one hour after the addition of the testing solution, denoted by ΔI % and defined by:

$$Δ I \% ( V ) = \frac{\vert {I}_{60 min} ( V ) \vert - \vert {I}_{PBS} ( V ) \vert}{\vert {I}_{PBS} ( V ) \vert} \times 100 \% . \qquad ( 1 )$$

Equation (1) shows that the first and last of the six I(V) curves were used to determine ΔI %, while the other four were plotted to illustrate the gradual change of the I(V) curves from 0 min to 60 min.

### 2.4. Biosensor Testing Protocols

First, the specificity of the vSiNW sensor against BSA was demonstrated, for which four combinations of functionalization and incubation were evaluated. For the functional sensors, ACE-2 was attached to the sensor surface, whereas for the control sensors, the ACE-2 immobilization was skipped, which means only BSA was used to terminate all the APTES molecules on the sensor. Then two types of incubation solutions were involved, each containing spike protein or BSA in the PBS. Combining the two types of functionalization and two types of incubation solutions, it resulted in four types of experiments. The positive test was conducted by testing the ACE-2-functionalized sensor with the spike protein (denoted by AS test or AS sensor), and three negative control tests included the ACE-2-functionalized sensor with the BSA (AB), the BSA-functionalized sensor with the spike (BS), and the BSA-functionalized sensor with the BSA (BB). The concentration of the spike was 7.5 μg/ml, and the BSA concentration was determined by maintaining the same density of molecules (in count/ml) as in the spike solution, which was approximately 2.5 μg/ml. In total, three AS sensors, three AB sensors, two BS sensors, and two BB sensors were tested. Then the concentration response of the sensor was demonstrated. All the vSiNW biosensors were ACE-2-functionalized, and the AS and AB tests for the specificity experiment corresponded to the spike concentrations of 7.5 μg/ml and 0 μg/ml, respectively. In addition, two other spike concentrations were tested, 0.06 μg/ml and 1.5 μg/ml, with one sensor at each concentration, and BSA was also added in those solutions to maintain the same density of molecules as the 7.5 μg/ml solution.

### 3. Results and Discussion

3.1. Modeling of the vSiNW Biosensor

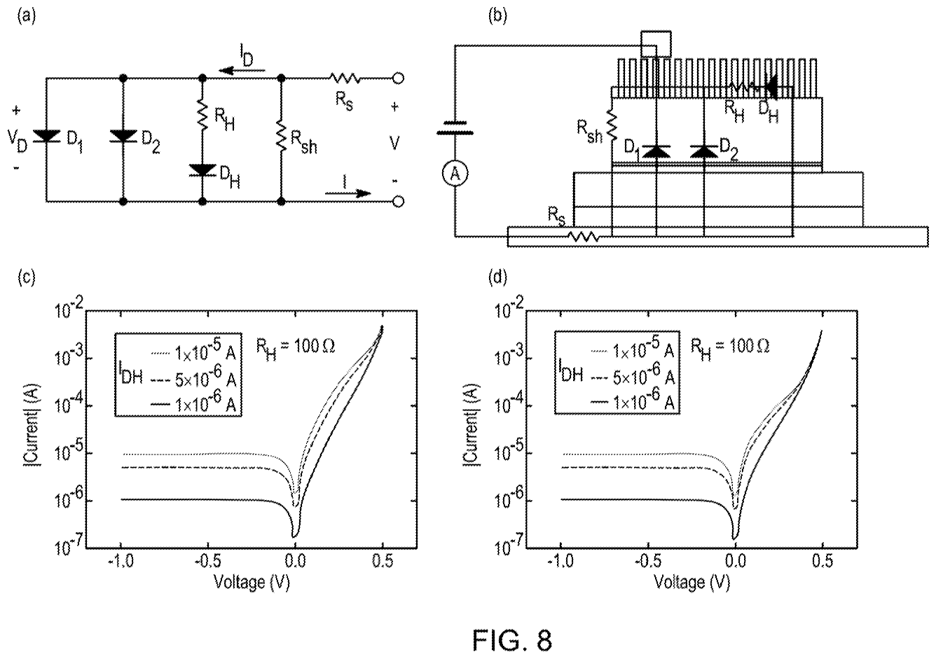

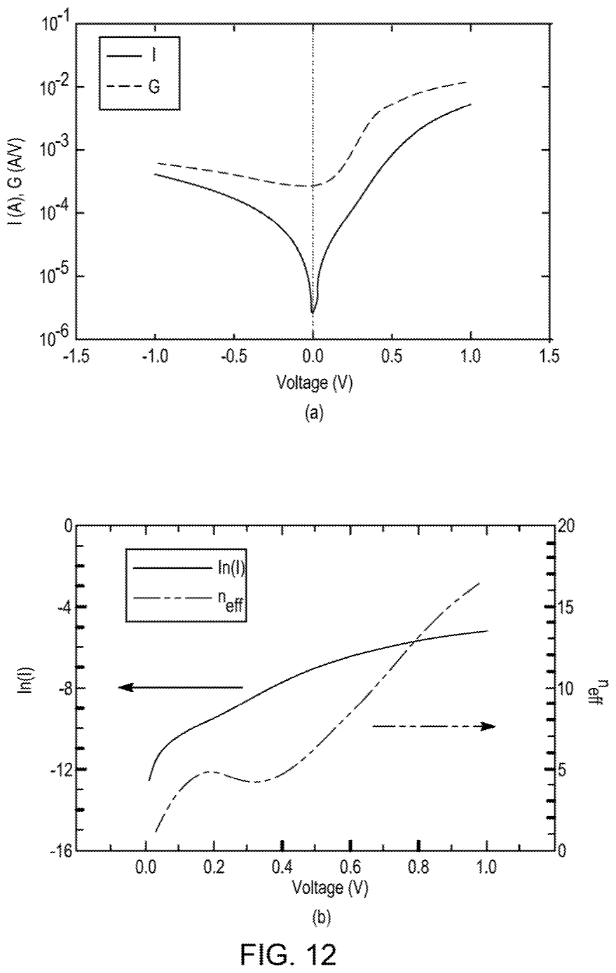

Devices with a p-n junction can be described using the Shockley equation for the diode model [41], from which the effective ideality factor n_{eff} can be approximated. However, using the measured I(V) curve of a vSiNW biosensor, it was found that the effective ideality factor n_{eff} as a function of V exhibits a “hump” that is much greater than two, under forward bias (FIG. 12). This effect indicates an enhanced front surface recombination that has been previously documented on nanotextured Si solar cells [42]. As a result, a “hump” diode, in addition to the traditional diffusion- and recombination-related diodes due to the p-n junction is induced, and the dark current model of device can be described using three diodes, referred as the three-diode model (FIG. 8(a)) [43, 44]. A possible source of each component of the three-diode model can be found in FIG. 8(b). The two diodes D₁ and D₂ are caused by the p-n junction-related current, and the Rₛ and Rₛₕ are the equivalent series and shunt resistances. The third diode D_{H}, the “hump” diode, is due to the surface recombination-related saturation current I_{0H}. The diode is also connected in series with an equivalent resistance R_{H} because it is away from the contact. Given the I(V) curve under high reverse and forward biases, the Rₛ and Rₛₕ can be found from the derivative of I(V) (see section S6 of Supporting Information), and the effects of Rₛ and Rₛₕ can then be eliminated from the I(V) curve by

$${V}_{D} = V - {IR}_{s} , \qquad ( 2 )$$

$${I}_{D} = I - \frac{{V}_{D}}{{R}_{sh}} , \qquad ( 3 )$$

where V_{D} and I_{D} are the voltage across D₁ and the sum of the current through all the diodes, respectively. The calibrated I(V) curve, I_{D}(V_{D}), is defined as the I_{D}-V_{D} curve, then ΔI % can also be calibrated by substituting I and V in Eq. (1) with I_{D} and V_{D} in Eq.s (2) and (3), which is denoted by ΔI_{D} %,

$$Δ {I}_{D} \% ( {V}_{D} ) = \frac{\vert {I}_{D 60 min} ( {V}_{D} ) \vert - \vert {I}_{D PBS} ( {V}_{D} ) \vert}{\vert {I}_{D PBS} ( {V}_{D} ) \vert} \times 100 \% . \qquad ( 4 )$$

Using ΔI_{D} %, the variations in Rₛ and Rₛₕ during experiment can be eliminated. However, there are still seven coupled variables in the model: the saturation current (I₀₁, I₀₂, or I_{0H}) and ideality factor (n₁, n₂, or n_{H}) for each diode (D₁, D₂, or D_{H}), and R_{H}. Regarding those variables, the following assumptions are made that due to the binding of ACE-2 and spike on the surface of the vSiNW sensor: (i) junction-related diodes D₁ and D₂ are not affected since the n⁺-emitter thickness is much greater than the Debye length of heavily doped Si [45], (ii) the ideality factor of D_{H} is fixed at 2.5 since it is dependent on the source of D_{H} [43, 44]. Then the only variables that may change the I(V) curve, are I_{0H} and R_{H}, whose effects on the simulated I_{D}-V_{D} curve are illustrated in FIGS. 8(c) and (d). From simulation, the characteristic effect of I_{0H} on the I_{D}-V_{D} curve is found to be such that as I_{0H} increases, induced by enhanced surface recombination, the I_{D}(V_{D}) increases, and the increase is significant only at V_{D} lower than a cutoff voltage V_{cutoff}. Besides, the V_{cutoff} is determined by R_{H} since the higher the R_{H}, lower the V_{cutoff}. Furthermore, the effect of R_{H} is found only significant at V>V_{cutoff}, therefore we can determine the cause of the change in I_{D}-V_{D} by checking the V range where the change is significant: the I_{D}(V_{D}) change at V_{D}<0 is from I_{0H}, whereas the I_{D}(V_{D}) change at V_{D}≥0.5 Vis from R_{H}.

In order to further demonstrate and find the major source of variation in the vSiNW sensor caused by a change in surface potential, a water-gate experiment was conducted by simulating the electrostatic potential of the spike protein using an external voltage source (see section S7 of Supporting Information), from which it has been found that the change in I_{0H} is dominating, and for negative (positive) surface potential, the I_{0H} increases (decreases) due to surface depletion (passivation) effect, and it is also roughly found that V_{cutoff}≈300 mV.

3.2. Testing of the vSiNW Biosensor and Calibration of I(V) Curves

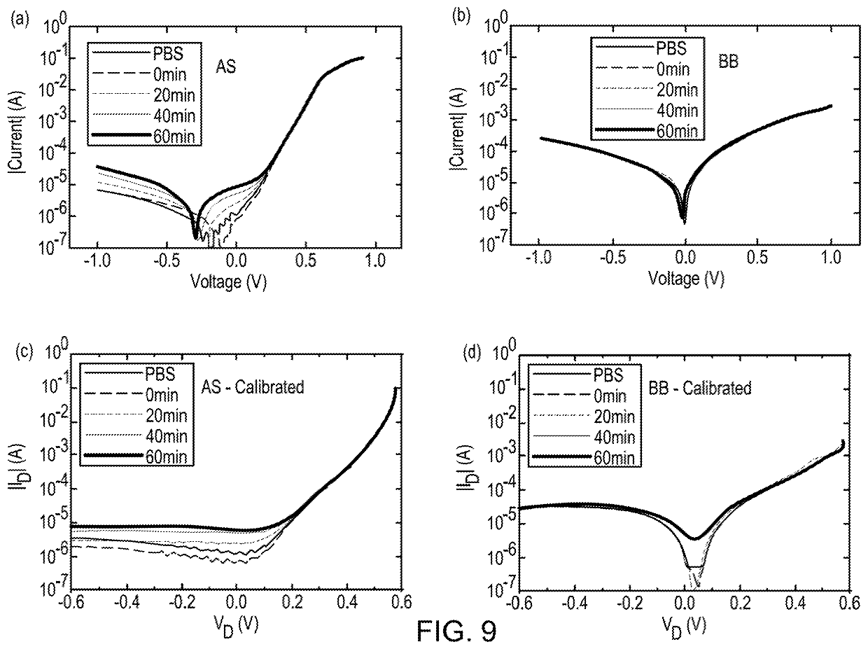

Given the analysis above, the vSiNW biosensor can then be tested with spike and BSA testing solutions. Theoretically, if the ACE-2 or (and) the spike is missing in the experiment, the non-specific binding of ACE-2-BSA, BSA-spike, or BSA-BSA will not induce randomly oriented electrostatic potential on the surface, hence the change of I(V) curves will be negligible. As for the specific binding of ACE-2 and spike proteins, net negative surface potential will be applied on the surface, which enhances the surface recombination effect of the n-type vSiNW and emitter and also increases I_{0H}. This effect has been found on the AS sensors, as the I(V) change of an AS sensor from I_{PBS} to I₆₀ₘᵢₙ is presented in FIG. 9(a). When the incubation time increases, the sensor shows a clear increase in I, and after 1 hour, an increase in I(V) of an order of magnitude is observed at V<0.3 V, indicating a substantial increase in I_{0H} induced by the negative surface potential. As a comparison, a BB sensor shows only has a small change in its I(V) curve after 1 hour in the BSA solution (FIG. 9(b)). The I(V) curves are then calibrated to eliminate the effects of Rₛ and Rₛₕ, as in FIGS. 9(c) and (d), from which the conclusion can be drawn that the I_{0H} has increased on the AS sensor whereas the increase is less significant on the BB sensor.

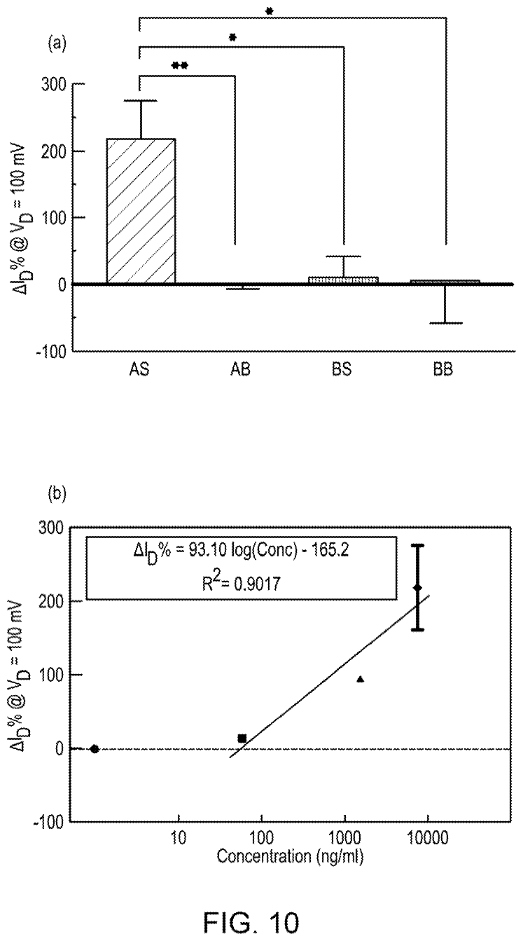

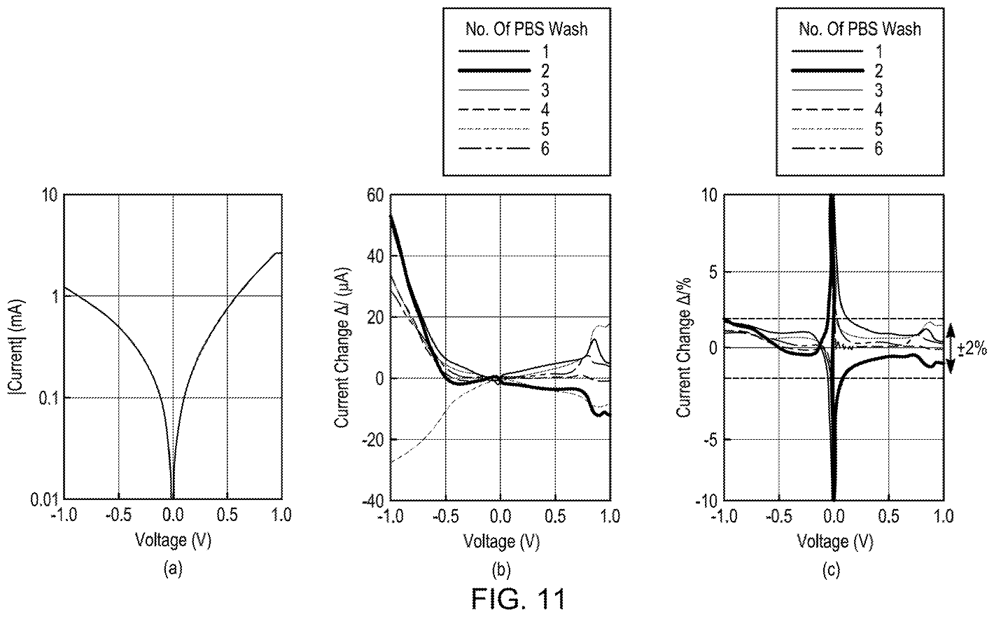

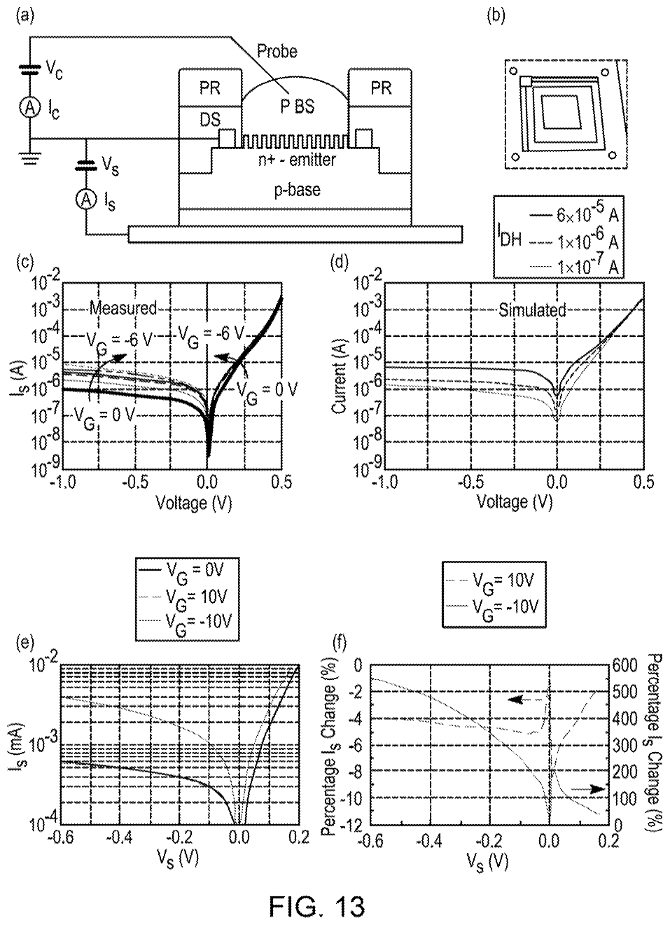

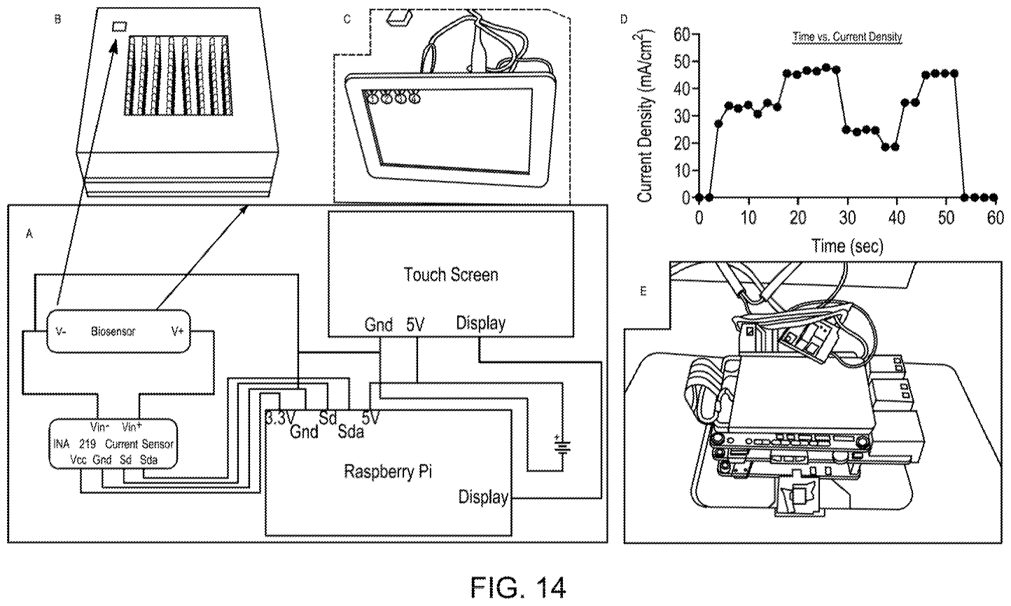

For each sensor in the experiment, ΔI_{D} % is calculated using Eq. (4). Although from FIG. 9 and the water-gate experiment (FIG. 13), all the values of ΔI_{D} % at V_{D}<300 mV can be used for demonstration, in this work, we choose to use ΔI_{D} % at V_{D}=100 mV. The main reason is that due to the limitation of the source meter, the low current values of sensors at −0.4 V<V_{D}<0.1 V are noisy (such as in FIG. 9(c)). Besides, the estimation of Rₛ and Rₛₕ could have uncertainties due to measurement errors, hence the ΔI_{D} % values at low biases are still more reliable than those at high biases. ΔI_{D} % at 100 mV of all the sensors for specificity experiment are presented in FIG. 10 Error! Reference source not found. (a), where the high specificity of the vSiNW biosensor is demonstrated clearly. The significantly low ΔI_{D} % for the negative controls with respect to the positive control indicates that non-specific binding cannot induce strong modulation of the biosensor's electronic response. The statistics of these controlled experiments confirm the high specificity response of the vSiNW biosensor for the spike protein when ACE2 is conjugated on the vSiNWs surface.

Furthermore, by testing at two more concentrations of the spike solution, ΔI_{D} % at 100 mV at those concentrations are plotted in FIG. 10(b). The data for 0 ng/ml corresponds to the AB functionalization scheme in FIG. 10(a), which is a good negative control for the sensor. By analyzing the other groups with spike concentrations higher than zero, a semi-logarithmic relation between the concentration and ΔI_{D} % is found, which has been theoretically demonstrated by Nair et al. The regression equation indicates a strong ΔI_{D} % response of 93.10% per decade increase of concentration, hence the sensitivity or the resolution of concentration is high in the semi-log region. The ΔI_{D} % is close to zero at 100 ng/mL or 575 pM (ng/ml to pM conversion is explained above in Section 2.1) of spike protein, which defines the limit-of-detection (LOD) for our biosensor. This LOD is significantly higher than some other reported works for ACE2 receptor-based biosensors and lower than others [13]. Therefore, we have successfully demonstrated a proof-of-principle for a vSiNW based biosensor platform for sensitive and selective detection of SARS-CoV-2. Future efforts will involve utilizing antibodies instead of ACE2 as the receptor for the spike protein given antibodies have many fold higher binding affinity to the protein than ACE2 [47], which would result in significantly improved sensitivity and LOD of our biosensor platform.

### 4. Conclusion

In this example, a vSiNWs-based biosensor for SARS-CoV-2 detection is demonstrated. The diode-type vSiNW biosensor with an area density of 10¹⁰ vSiNWs per cm² and average vSiNW length of ˜350 nm is fabricated using a scalable MACE process. ACE-2 is chosen as the functionalization protein, with successful assay tests showing specific binding of ACE-2 and SARS-CoV-2 spike proteins. A three-diode model of the sensor is described, which indicates that the I(V) characteristics of the sensor can be used to detect biomolecules that exhibit electrostatic polarity. Sensitive and specific detection of the SARS-CoV-2 spike protein utilizing the vSiNW biosensor is presented and confirmed. This work demonstrates capabilities of the vSiNW biosensor platform for use in point-of-care (POC) SARS-CoV-2 detection.

### Example 2—Sensitive and Specific Detection of Estrogens Featuring Doped Silicon Nanowire Arrays

Estrogens and estrogen-mimicking compounds in the aquatic environment are known to cause negative impacts to both ecosystems and human health. In this example, the vertically-oriented silicon (Si) nanowire (NW) array-based biosensor is used for low-cost, highly sensitive and selective detection of estrogens. The Si NW arrays were formed using an inexpensive and scalable metal-assisted chemical etching (MACE) process. A p-n junction design for the biosensor was used and functionalized via 3-aminopropyltriethoxysilane (APTES) based wet-chemistry to bond estrogen receptor-alpha (ER-α) to the surface of the Si NWs. Following receptor conjugation, the biosensors were exposed to increasing concentrations of estradiol (E2), resulting in a well-calibrated sensor response (R²≥0.84, 1-100 ng/ml concentration range). Fluorescence measurements quantified the distribution of estrogen receptors across the Si NW array compared to planar Si, indicating an average of seven times higher receptor presence on the NW array surface. We tested the biosensor's target selectivity by comparing it to another estrogen (estrone [E1]) and an androgen (testosterone), where we measured a high positive electrical biosensor response after E1 exposure and minimal after testosterone. The regeneration capacity of the biosensor was tested following three successive rinses with phosphate buffer solution between hormone exposure. Most traditional horizontally-oriented Si NW biosensors report electrical current changes at the nanoAmperes (nAs) level that require prohibitively expensive measurement equipment; our biosensor exhibits current changes in the microAmperes (μAs) scale, demonstrating up to 100-fold electrical signal amplification, thus enabling signal measurement using inexpensive equipment. The highly sensitive and specific biosensor developed here allows for low-cost, portable, field-deployable biosensors that can detect estrogenic compounds in waterways in real-time.

### 1. Nanofabrication Process Steps for the Si NW Biosensor

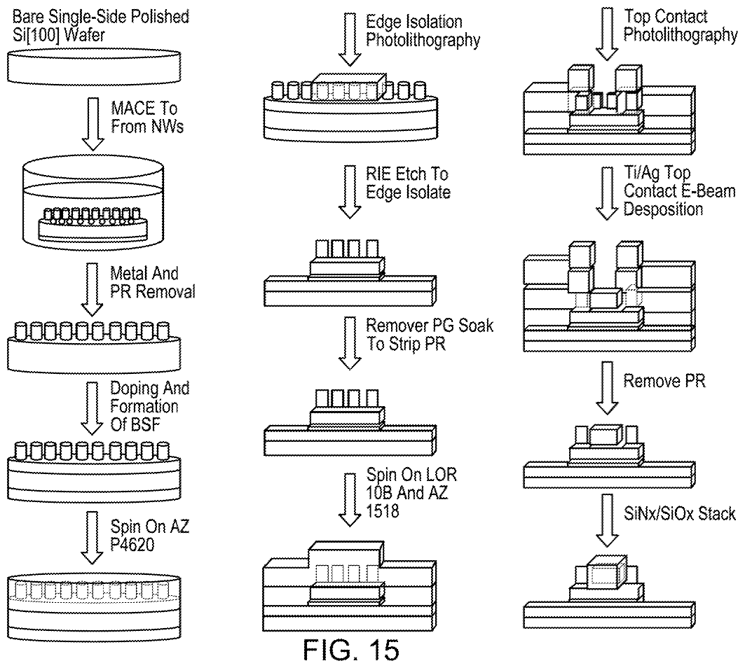

The summary schematic representing the nanofabrication process steps are shown in FIG. 15. The details of each of the process steps are discussed next.

### 1.1. Fabrication of Si NWs Using Silver Nanoparticles

We purchased single-side polished, two-inch diameter, 280-μm thick, boron-doped (1-10 Ω-cm resistivity), monocrystalline silicon (Si)<100>Czochralski (CZ) wafers from University Wafer. These Si wafers are first thoroughly cleaned using the Radio Corporation of America (RCA) Standard Cleans (SC)¹ and hydrofluoric acid (HF). The RCA SC-1 solution is made of deionized (DI) water, ammonium hydroxide (NH₄OH), and hydrogen peroxide (H₂O₂) in a 5:1:1 ratio. The solution is heated up to and maintained at around 70° C., and the wafers are submerged for 10 minutes. This basic mixture removes the organic contaminants on the wafer. The wafers are then immersed in a 10 vol % solution of HF to strip the oxide, then rinsed in DI water. RCA SC-2 solution is made using DI water, hydrochloric acid (HCl) and H₂O₂ in a 6:1:1 volume ratio, mixed, and heated to around 70° C. The wafers are submerged and cleaned in the solution for 10 minutes and finally rinsed in DI water. This removes the remaining ionic residues and slightly passivates the wafer to protect the substrate surface from further contamination.

To prevent the backside of the Si wafer from etching, the back of the Si substrates is coated with photoresist (PR) AZ P4620 and baked for 10 minutes on a hot plate at 120° C. Another HF clean is performed, and the wafer is rinsed in DI water. To increase the hydrophilicity of the surface and ensure uniform etching, the wafers are submerged in 30 vol % H₂O₂ until the surface is completely oxidized, about a minute or so. A one-step silver (Ag) MACE process is performed by submerging the substrate into a room temperature solution containing silver nitrate (AgNO₃), HF, and DI water for 5 minutes. For the target Si NW length of around 500 nm, a 100 mL mixture is composed of 17 mL of HF, 70 mL of DI water, and 13 mL of 154 mM AgNO₃, resulting in a 5M HF and 20 mM AgNO₃ concentration solution. The solution is stirred continuously as the etching occurs. After etching, the backside PR is removed with an acetone and isopropanol (IPA) rinse, followed by a dip in DI water. The residual Ag nanoparticles at the bottom of the Si NWs, are removed by submerging the substrate in room temperature nitric acid (HNO₃) for 3 minutes and rinsed in DI water again. The Si NW samples are then immersed in a buffered oxide etchant (BOE) for 2 minutes to further clean the samples, rinsed in DI water and gently dried using a dry nitrogen (N₂) gun.

### 1.2. Simultaneous Front Doping and Back-Surface-Field Formation

A nanofabrication process was developed by our team to simultaneously achieve frontside n+ doping and p+ back-surface-field (BSF) formation. The frontside n+ emitter is formed by proximity doping using ammonium dihydrogen phosphate (ADP)² as the spin-on-dopant. ADP solution is prepared by mixing 0.85 wt % ADP and DI water. The source wafers are prepared for proximity doping: they are first cleaned in RCA SC-2 cleaning solution, then the oxide is removed with a dip in HF solution. Finally, the source wafer is dipped in Nanostrip solution for 10 minutes at room temperature to increase the hydrophilicity of the surface by forming a dense oxide film on the surface to help the ADP solution spread uniformly across it. The ADP solution is then spin-coated on the polished side of the Si source wafer at 2000 rotations per minute (rpm) with a 200 rpm/s ramp for 1 minute using a Laurell Technologies Spin Coater and baked for 8 minutes at 100° C. on a hot plate.

The backside is coated with a thin layer of aluminum (Al) paste that is carefully applied, using a scraper as a squeegee to clean off the excess and pre-baked at 180° C. for 5 minutes on a hot plate. Al is a known p-type dopant of Si; therefore the presence of Al paste ensures a robust p+ BSF. The Al also serves as the back contact of the biosensor. The final setup is then assembled on a quartz plate to be inserted into an MTI Corporation EQ-RTP-1000D4, a rapid thermal annealing (RTA) furnace. The Si source wafer is placed ADP side up on the quartz plate, while the Si substrate is placed NW side down, separated by 500 μm Si spacers. This setup is then placed into the RTA chamber, which is then pumped down. N₂ gas is then vented into the chamber at a 100 sccm flow, and the pump is adjusted so that the pressure in the chamber is maintained at 1 atm (760 Torr). The chamber temperature is ramped up to 950° C. in 10 minutes and maintained at that temperature for 10 minutes. This process results roughly in a front junction depth of 0.7 μm, which is deeper than the NW length, and ensures that the junction is formed under the NWs. Afterwards, the sample is removed once the chamber has naturally cooled to room temperature. The phosphosilicate glass (PSG) residue that is formed on the surface of the doped samples is removed by a 30 second dip in BOE. The final Rₛₕₑₑₜ of the Si NW samples is then measured using a Signatone S-302 4-point Resistivity Probe in our lab.

### 1.3. Edge Isolation

To prevent shorting out the sensor, edge isolation is performed on each p-n junction device. PR AZ P4620 is spin-coated on the front at 4000 rpm with a 4000 rpm/s ramp for 1 minute and dried on a hot plate at 100° C. for 4 minutes. Since the developer will etch Al, the backside is also coated in a layer of AZ P4620. Since AZ P4620 is a thick resist, there may be buildup on the edges of the sample where the photoresist is more elevated than the rest of the surface, called edge beads. This can behave as an unwanted spacer between the substrate and the mask, as well as lead to cracking or damaging the substrate. To remove any edge beads that have formed, a cotton swab dipped in acetone is used to smooth out the edges.

To ensure that the PR has sufficient water content when photolithography is performed, the samples are left on the wet bench for 20 minutes. Rehydration is an important step to guarantee a high development rate. At this step, the whole wafer is cleaved into quarters in anticipation for one sensor per quarter wafer. Afterward, the samples are then exposed to the pattern on an OAI Mask Aligner Model 800 for 24 seconds. The substrate is developed in AZ400K developer 1:4 developer for at least 4 minutes. The exposed pattern is an 11.5 cm by 11.5 cm square in the middle of the Si substrate.

The excess PR is rinsed off with more AZ 400K 1:4 and DI water to guarantee a clean edge. The sample is then etched by reactive ion etching (RIE) of Si using an Oxford Instruments RIE NGP80 machine. The dry etching is performed for 1 minute using a combination of tetrafluoromethane (CF₄), O₂ and Ar gases. After etching, the PR is stripped by soaking in MicroChem Remover PG heated to 80° C. for 5 to 15 minutes until the surface is clean. The samples are then rinsed in IPA and gently dried with an N₂ gun. A final Rₛₕₑₑₜ value is measured before front contacts are created.

### 1.4. Top Contact Patterning and Metal Deposition

Top contact patterning is done by first spin coating on two PRs. A lift-off resist (LOR) 20B is first spun on at 3000 rpm for 1 minute to make the PR easier to remove after metal deposition and dried on a hot plate at 150° C. for 4 minutes. The backside is again coated in AZ P4620 and dried using the same instructions as mentioned previously. The front side is then coated with a layer of AZ 1518, spun on at 3500 rpm for 1 minute and dried on a hot plate at 100° C. for 3 minutes. The sample is exposed for 8 seconds to the contact pattern using photolithography and submerged in AZ 400K 1:4 developer.

To facilitate smoother metal deposition, oxide removal of the exposed Si area is then performed by dipping the samples for 30 seconds in BOE. The samples are thoroughly dried under an N₂ spray. Using an Angstrom Engineering 6-pocket E-Beam Evaporator, 50 nm of Ti and 1 μm of Ag are deposited to form a top metal contact. To lift off the PR, the samples are then submerged in 80° C. Remover PG while stirred and left overnight if necessary due to the thickness of the metal. After the samples are removed from the Remover PG, the samples are rinsed in IPA and DI water to ensure the removal of any remaining PR.

### 1.5. Dielectric Protection of Top Metal Contact

To protect the front metal contact during future wet biofunctionalization process steps, two stacks of alternating silicon nitride (SiNx) and silicon dioxide (SiO₂) layers are deposited on the front surface. The dielectric pattern is designed to leave an open area in the center of the surface for the exposed NWs. The Si substrate are spin-coated with PR using the same layered process for top contact patterning. This time, the sample is exposed to a different mask that ensures everything except for the center of the sensor and the contact pad will be covered by the dielectrics. The samples are inserted into an Intl Vac—Nanochrome I Sputterer, where 50 nm of SiOx and 100 nm of SiNx are deposited alternately until two layers of each dielectric are placed down. We selected the dielectric stack thickness to ensure around 10⁻⁵ g/m²/day water vapor transmission rate (WVTR), which was found to be sufficiently low to form a barrier of protection against liquids that may loosen the metal contact from the Si surface and render the sensor non-functional. The PR is lifted off, after the dielectric deposition, by submerging the substrates in 80° C. Remover PG overnight. The fabricated sensors are then washed with IPA and DI water, dried with N₂ gun, and ready for biofunctionalization.

### 2. Biofunctionalization of the Si NW Biosensor for Hormone Detection

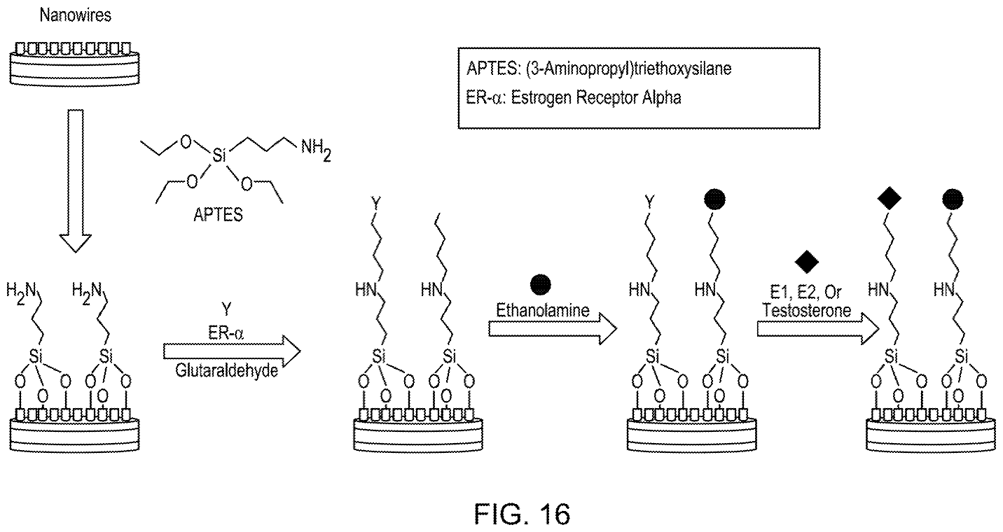

The summary schematic representing the biofunctionalization steps are shown in FIG. 16. The details of each of the process steps are discussed next.

### 2.1. Sensor Biofunctionalization Chemistry and Process Details

The Si NW biosensor surface is first functionalized using 10 mL 2% APTES solution in ethanol/H₂O (95/5, v/v) for 2 hours, then the surface is rinsed with ethanol, and dried using a N₂ gun. Next, the biosensor surface is functionalized using 10 mL 2.5% glutaraldehyde solution in DI water for 1 hour, rinsed with DI water, and dried via a N₂ gun. This bifunctional linker contains two aldehyde terminals, which enables one end to bind to the amine-terminated APTES and the other end to immobilize the ER-α protein. Following drying, the ER-α protein is covalently bound onto the surface of the Si NWs by incubating the sensor in 2 mL 10 μg/mL ER-α protein for 6 hours. The unbound ER-α protein is then removed with a 0.01X PBS buffer wash and the surface is dried with N₂. The estrogen receptor-functionalized surface is then passivated with 10 mL 100 mM ethanolamine in 0.01X PBS buffer to minimize non-specific binding for 1 hour, followed by drying with N₂.

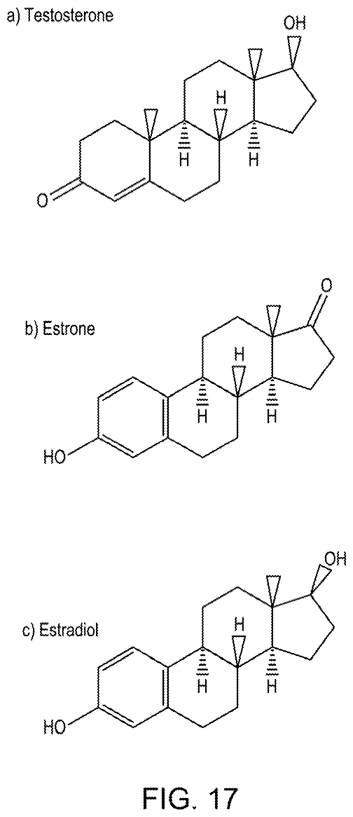

Following functionalization with ER-a, we tested the biosensors with three different hormones: estrone (E1) and 17ß-estradiol (E2), and testosterone (a non-estrogen androgen hormone that should not bind to ER-a) at concentrations of 1-10,000 ng/ml. FIG. 17 shows the stereochemical structures of the three hormones tested with our biosensor. 5 mL 0.01X PBS buffer containing target hormone is introduced onto the post-receptor Si NW surface, and the sensor is incubated for 1 hour. After incubation, the surface is dried with N₂ gun and ready for electrical current measurements. All the steps are performed on an orbital shaker table (Cole-Parmer) to ensure uniform coverage of the functionalization agents on the Si NW sensor surface. To regenerate the biosensor, the biosensor surface is washed with pure buffer solution (0.0001X PBS buffer) for 5 minutes.

### 2.2. Use of Aminopolyethylene Glycol (Amino PEG) Versus Ethanolamine to Block Non-Specific Binding

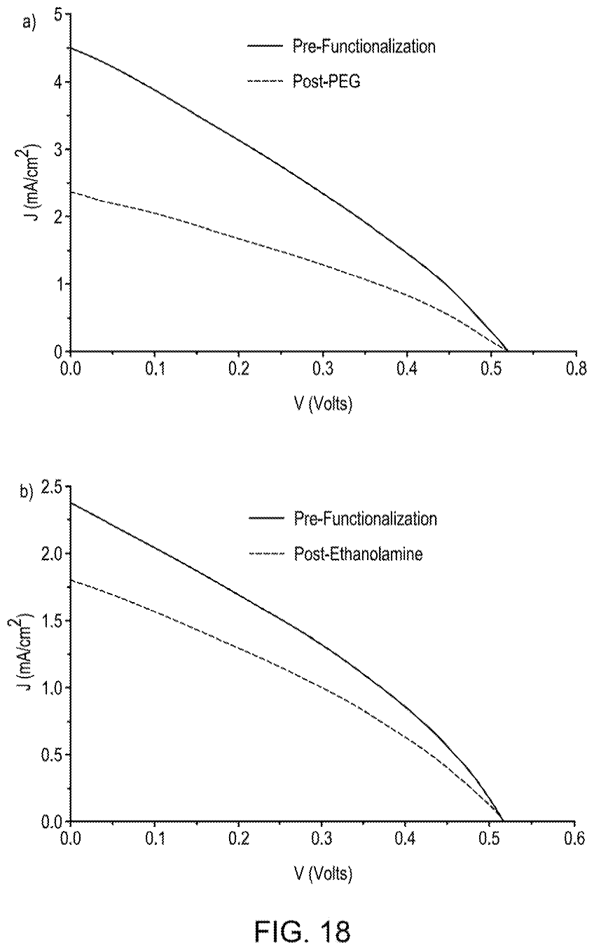

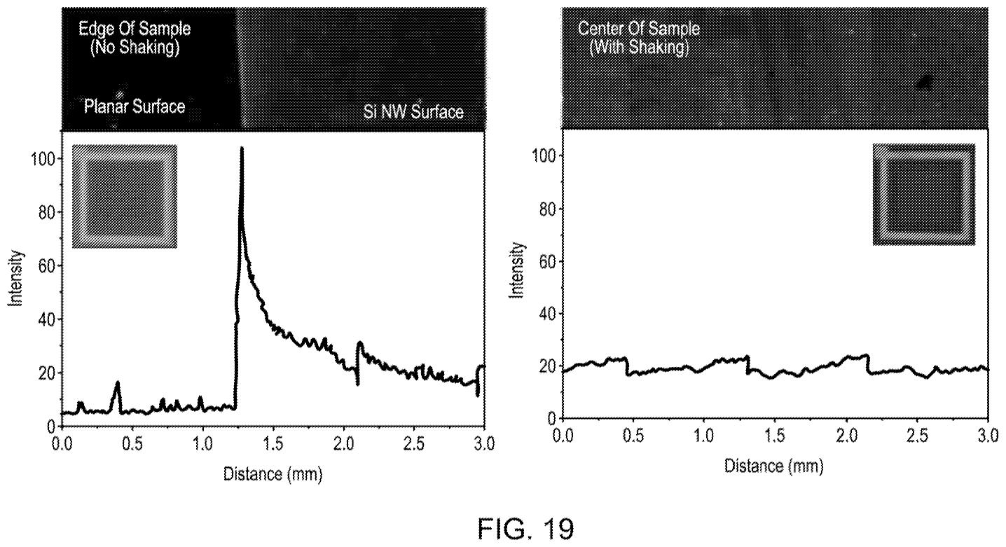

After functionalization, there is no guarantee that every amine has an estrogen receptor attached to its end. Therefore a molecule is introduced in the biofunctionalization process to block all the unlinked amines. In our initial experiments, we passivated the unlinked amines with a protein called methoxypolyethylene glycol amine (amino PEG). The molecular weight of amino PEG depends on the number of polyethylene glycol (PEG) molecules conjugated together. However, even the smallest iteration of one PEG molecule is far too large for our sensor surface and possibly interfered with the receptor binding. We found this because, after the sensor incubation in amino PEG, we measured a significant degradation in the effectiveness of the biosensor, as seen in FIG. 18. Therefore to prevent the biosensor response from degrading after functionalization, we used ethanolamine as an alternative passivant, as both a smaller molecule and an inexpensive alternative. This switch significantly reduced the degradation of current density-voltage (J-V) response of the biosensor, as shown in FIG. 18(b), where after amino PEG functionalization the current density dropped by around 2 mA/cm², while with ethanolamine the drop was around 0.6 mA/cm².

### 2.3. Fluorescence Imaging to Confirm Uniform Distribution of Receptor on the Biosensor Surface

We tested the Si NW biosensor surface with fluorescence imaging to confirm uniform distribution of the biofunctionalized ER-α receptor over the surface. For preparing the sensor for fluorescence imaging, the biosensor was functionalized up to the ER-α receptor as explained in Section S.3.1., the Si NW surface was stained with ER-α protein monoclonal antibody (Thermo Fisher product #MA5-13191) at a dilution of 1:750 in 0.01X PBS buffer solution overnight at 4° C. The sample was then incubated with DyLight 488-conjugated goat anti-mouse IgG secondary antibody (Thermo Fisher product #35502) at a dilution of 1:500 in 0.01X PBS buffer solution for 1 hour at room temperature, then the sample surface was dried using a N₂ gun. All fluorescence data were collected on the Zeiss LSM 710 confocal microscope.

As shown in FIG. 19(a), receptors did not spread uniformly on the surface when the sample was not agitated during functionalization. The sharp increase in intensity shows all the receptors clustered at the edges of the samples and very little of the receptors were present in the center of the sample, which could result in decreased the responsivity of the sensor. If the receptors were not spread evenly over the surface, they will compete for the hormones and therefore the sensor signal change will be lower. To resolve this, a shaking table was utilized to slowly shake the beaker holding the biosensor while it was immersed in the receptor solution. This helped to evenly distribute the receptors across the sensor surface. After being stained by fluorescent antibody, the receptors are much more evenly distributed, as shown in FIG. 19(b). The entire sensor surface is at the same fluorescent intensity instead of the high intensity spike seen on the edge of the sensor in the no shaking case.

### 2.4. Current Density Versus Voltage (J-V) Measurements

The current versus voltage (I-V) measured were performed using a Keithley 2400 Source meter. The front of the biosensor was contacted with a tungsten needle attached to a Quater XYZ 300 TL micro positioner, and the back of the sensor sat on an Oriel Instruments Basic PVIV vacuum chuck. Both the front and back contacts of the biosensor were then connected through cables to the source meter. A PC Lab View program processed the I-V data measured by the source meter connected to the biosensor. The electrical current values were converted to current density (J), using the biosensor active area. J-V curves were measured in the dark before and after the exposure of E2 to eliminate any external influences on the biosensor. For consistency, the J value at 600 m V was selected so that the bias voltage was high enough that the biosensor was effectively “turned on” and sufficient electrical current was flowing through the device.

### 2.5. AJ Calculation Methodology

After the biosensor is functionalized with the receptor, a JV curve is first taken as a baseline. After the introduction of estrogen or any other hormone to the biosensor, another JV curve is taken and compared to the baseline data. Using these two curves, a change in current density (ΔJ) is calculated:

$$Δ J = {J}_{Post - Estrogen} - {J}_{Post - ER α} .$$

### NW Array Doping Density and Biosensor Sensitivity

We fabricated the biosensors with varied doping levels and measured the NW sensor response to a range of E2 concentrations (1 ng/mL, 10 ng/mL, and 100 ng/mL). The biosensor had the largest current density response when the NW array doping was low, which resulted in effective sheet resistivity (Rₛₕₑₑₜ) values in the 1000-1200 Ω/sq range. Doping levels are directly related to sheet resistivity, where a high Rₛₕₑₑₜ value corresponds to a low doping concentration, and vice versa. The effects of both E2 concentration and NW array Rₛₕₑₑₜ were evaluated by the change in biosensor current density response. We also compared current density change of our vertically-oriented Si NW array biosensors and the conventional horizontally-oriented Si NW biosensors to demonstrate up to 100-fold electrical signal amplification in our biosensors relative to the conventional ones.

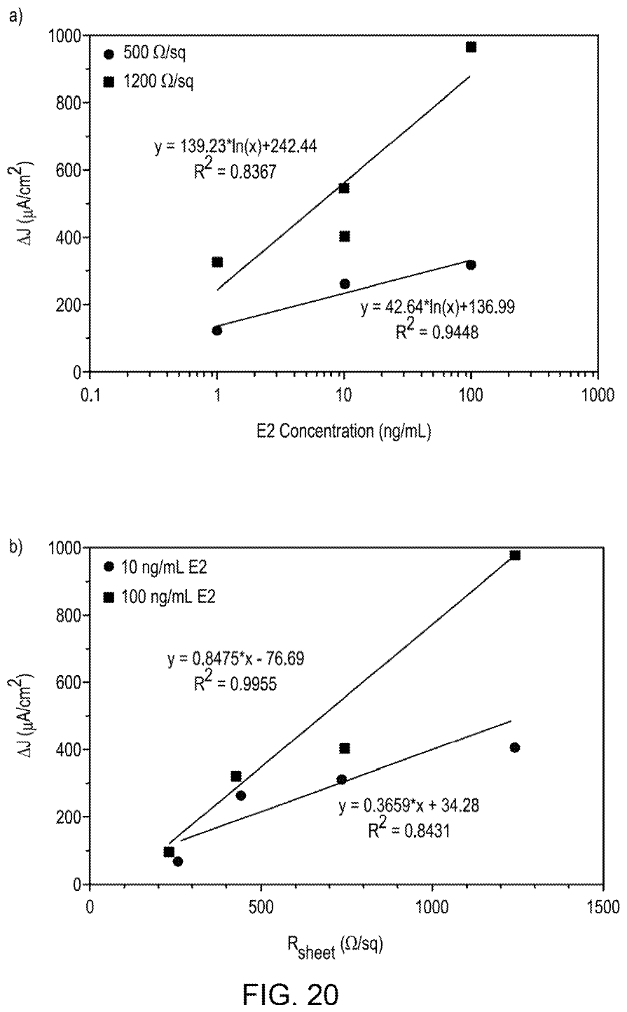

We also quantified the relationship between changes in current density (ΔJ) and E2 concentrations for two different Rₛₕₑₑₜ values (FIG. 20a). We tested sensors with two different sensor surface sheet resistivities (nominal value of 500 Ω/sq and 1200 Ω/sq, respectively). These results demonstrate a strong positive relationship between current density change and E2 concentration (R2≥0.84; FIG. 20a). When the biosensor was highly doped (low Rₛₕₑₑₜ, 250 Ω/sq), the change in ΔJ was at least seven times less than a low doped biosensor (high Rₛₕₑₑₜ, 1200 Ω/sq) for the same E2 concentration (FIG. 20b). Two different concentrations of E2, 10 ng/ml and 100 ng/mL, were exposed to biosensors at varied levels of doping. Linear regression fits between Rₛₕₑₑₜ and AJ for the two concentrations (R2≥0.84; FIG. 20a) The biosensors with Rₛₕₑₑₜ of around 1200 Ω/sq exhibited the largest AJ after exposure to E2 as well as the largest current density difference between the 10 ng/mL and 100 ng/ml concentrations. No data exists for higher Rₛₕₑₑₜ values because the biosensors were unable to generate a measurable electrical signal with such low doping levels. Nevertheless, there is a lower limit to doping the Si NWs because extremely low doping prevents the p-n junction from being formed, and the device therefore will not be electrically active. We note that the estrogen levels tested herein are still orders of magnitude higher than the typical natural aquatic conditions; thus, further research to lower the detection limits is needed.

Biosensor sensitivity is known to be affected by the doping of the NWs. Specifically, when the biosensor surface is highly doped, detection of the target molecule decreases due to screening effects and the recombination rate. As the concentration of carriers increases, an electrostatic effect known as the screening effect occurs where a carrier repels other carriers and creates what is known as a “screening hole” around itself. The electric field within the screening hole is cancelled and leads to a lower current density response. Furthermore, as NW doping increases, the carrier recombination rate increases resulting in a decrease in minority carrier diffusion length and an overall lower current density change.

Our vertically-oriented Si NW biosensor exhibited a substantially higher electrical change compared to traditional horizontally-oriented Si NW biosensors. When reporting on NW signal responses, most biosensors report electrical current changes at the nA level. Such small electrical signals require expensive measurement equipment (>$10,000). In comparison, our biosensor has electrical current changes in the microAmperes (μA), demonstrating up to 100 times electrical signal amplification relative to traditional NW biosensors. This amplification enables measurement of our sensor signal using inexpensive equipment (˜$200), emphasizing the advantages of our vertical NW array-based biosensors and the potential to use the biosensors in a non-standard lab setting, such as in water streams for real-time data collection.

Thus, the Vertically-Oriented Silicon Nanowires Array-based Biosensor Platform may be used for analytes such as hormones. In addition, because high sheet resistivity Rₛₕₑₑₜ results in high sensitivity sensor response, the identification of this relationship may be used to improve biosensors. In particular, the Rₛₕₑₑₜ may be selected or designed specifically for the purpose of increasing sensitivity of sensor response.

### Example 3—Vertically-Oriented Silicon Nanowires Array-based Biosensor Platform for Label-Free Detection of Protein and ctDNA Colorectal Cancer Biomarkers

Monitoring levels of protein and circulating tumor DNA (ctDNA) biomarkers in liquid biopsy samples is integral to the care of colorectal cancer patients. Here we report that appropriately functionalized vertically-oriented silicon nanowires array-based biosensor platform enables label-free detection of carcinoembryonic antigen (CEA), carbohydrate antigen 19-9 (CA19-9), and BRAFV600E ctDNA mutation. Our results confirm a sensitive response using the novel biosensor platform that can detect clinically relevant concentrations of CRC biomarkers, CEA (<2.5 ng/ml), CA19-9 (<37 U/mL), and BRAFV600E (<11.52 μM).

1. Functionalization of vSiNW-Diode Biosensors with Monoclonal Antibodies (Anti-CEA, Anti-CA 19-9) (MoAb)

Before conjugation of the bioentities on the vSiNW-diode biosensors, the sensors were placed in 30 ml beakers and sterilized under UV light for 30 minutes. Following this, biosensors were rinsed with 5 ml of acetone and isopropyl alcohol (Sigma Aldrich), then NANO pure Diamond™ water (Barnstead). Solvents were removed and then sensors were incubated with 10% (3 aminopropyl) triethoxysilane (APTES) solution (Acros Organics), for 1 hour at room temperature to activate the surface with an amine-terminated moiety. Next 1-ethyl-3-(3-dimethylaminopropyl) carbodiimide/N-hydroxysulfosuccinimide (EDC/sNHS) (ThermoFisher Scientific) chemistry was employed to covalently link monoclonal antibodies (MoAB); either human IgG anti-CEA (anti-CEA, clone 1105, Thermofisher Scientific) or human IgG anti-CA 19-9 (anti-CA 19-9, clone 116-NS-19-9, Thermofisher Scientific) by incubating vSiNW-diodes with 1 ml antibody solution (0.05 M EDC, 0.02 M sNHS, 10 μg ml⁻¹ MoAB) for 3 hours. Sensors were then washed with 0.05% tween-20 in 1× phosphate-buffered solution (PBS) and next with a 10-μg ml⁻¹ solution of bovine serum albumin (BSA).

### Electrical Characterization Setup

The current (I) versus voltage (V) measurement, denoted by I-V measurement, was performed by connecting the biosensor with a Keithley 2400 sourcemeter unit (SMU) through cables. The front Ag contact was contacted with a tungsten needle electrode attached to a positioner with a cable. The back of the sensor was placed on an Oriel Instruments Basic PVIV vacuum chuck with copper (Cu) tapes inserted in-between to cushion the sensor from vibration and enhance conduction. A PC Lab View program was utilized to process the I-V data measured by the SMU. The integration time was set up as 1 power line cycle and the V scanned from 0 V to 1 V under reverse bias. Reverse current values were compared to monitor the change in saturation current caused by the modulation of vSiNWs due to the binding of polar biomolecules.

Raw data for ΔI % as a function of CEA concentration in ng/ml utilizing vSiNWs-diode's I-V data. ΔI % is computed at 600 mV to eliminate effects of shunt and series resistances of the diode.

| Concentration | |
|---|---|
| (ng/mL) | ΔI % |
| 1 | −48.21 |
| 10 | −79.1, |
| | −86.25 |
| 10,000 | −120 |

Raw data for ΔI % as a function of CA 19-9 concentration in U/mL utilizing vSiNWs-diode's I-V data. ΔI % is computed at 600 mV to eliminate effects of shunt and series resistances of the diode.

| Concentration | |
|---|---|
| (U/mL) | ΔI % |
| 10 | −0.34796, |
| | 0.791952 |
| 100 | −1.9932 |
| 1000 | −1.96259, |
| | −1.56156 |

1. Functionalization and characterization of planar Si and vSiNWs with synthetic BRAFV600E capture and Alexa Fluor 488 tagged target DNA

The DNA functionalization of planar Si and vSiNWs samples started with placing the samples in 30 ml beakers and sterilizing them UV light for 30 minutes. Next the samples were washed with 1 mL acetone, and 1 mL isopropyl alcohol (Sigma Aldrich), and rinse in 1 mL DI water three times to ensure removal of contaminants. Next the samples were incubated in 1 mL of 10% APTES (Acros Organics) solution in toluene for 1 hour at room temperature. After the incubation, the samples were rinsed in 1 mL toluene, followed by 1 mL of methanol, and rinsed with 1 mL DI water three times to ensure removal of contaminants. Next the samples were incubated in glutaraldehyde (Sigma Aldrich) solution (2.5% diluted in 1×PBS solution) for 30 mins and rinsed in 1×PBS three times. Following this, the vSiNW samples were incubated in 1 mL of 20 nM primer solution containing the capture DNA sequence: 5′-AAT AGG TGA TTT TGG TCT AGC TAC AGT-3′ (Invitrogen) for 1 hour at room temperature and rinsed with DI water. Next the vSiNW samples were incubated in 1 mL of 20 nM primer solution containing the target DNA sequence: “/5Alex488N/ACT GTA GCT AGA CCA AAA TCA CCT ATT-3 (Invitrogen) for 1 hour at room temperature and rinsed with DI water. Finally, the samples were washed with ethanolamine (Sigma Aldrich) to block unreacted glutaraldehyde sites and rinsed in DI water. Following the DNA conjugation steps, confocal microscopy (Zeiss LSM 710) imaging was performed on various samples, and the captured images were analyzed utilizing ImageJ. Confocal images for the analyzed samples were used to compare planar Si conjugation and vSiNW surface conjugation to demonstrate the benefits of using NW-etched surfaces. To confirm that in fact the fluorescence signal was from capture-target DNA hybridized surfaces, we also measured the samples after denaturing them in hot water kept at 90° C.

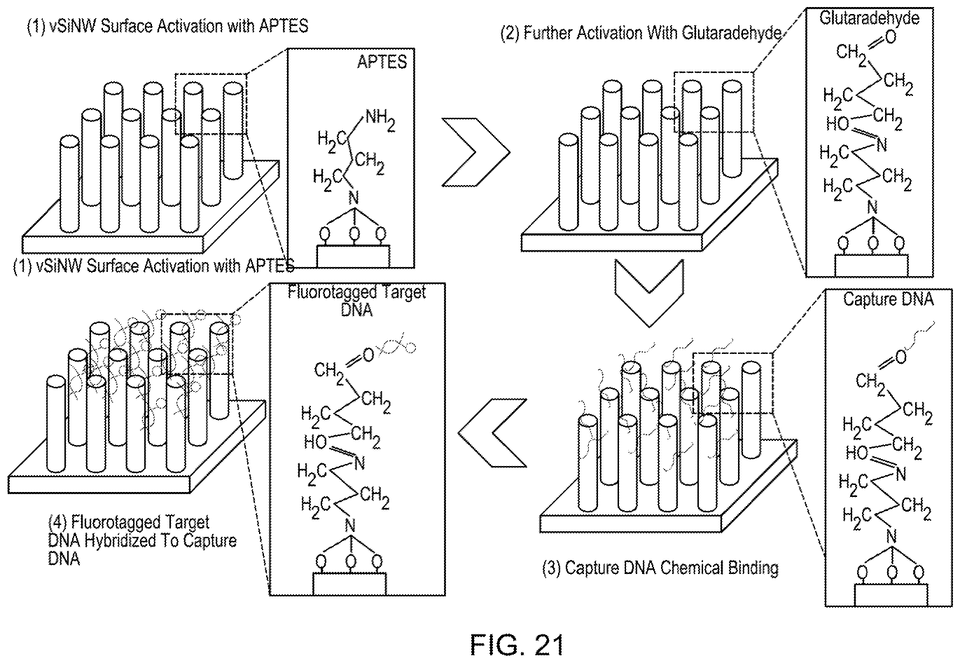

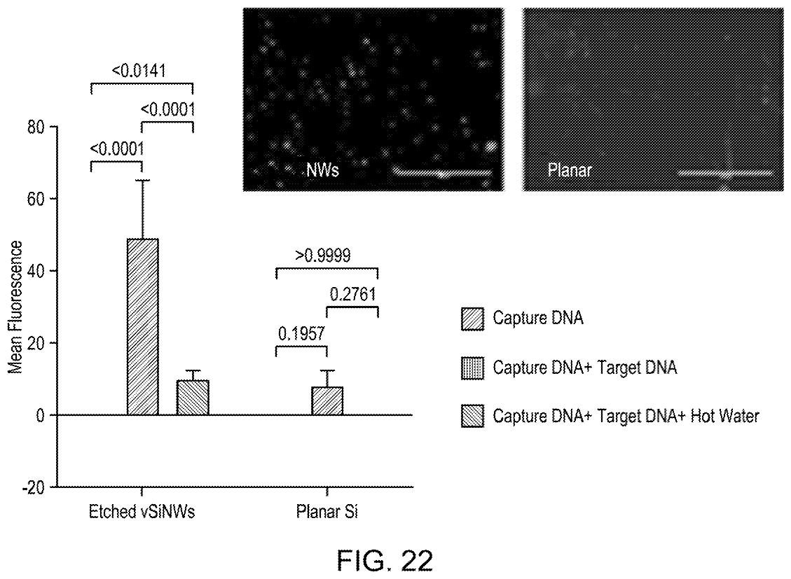

Thus, the clinical relevance of the vSiNWs based biosensor platform was demonstrated by conjugating hybridized synthetic BRAFV600E DNA, a common CRC ctDNA biomarker on planar and vSiNWs samples. FIG. 21 illustrates the procedure used to immobilize the synthetic DNA strands onto the surface-bounded APTES units. The planar and vSiNWs samples were incubated in APTES solution, which resulted in amino groups presenting as terminal units from the Si surface. FIG. 21 provides schematics illustrating the self-assembly processes for the immobilization of capture DNA and hybridization of the target DNA on the vSiNWs surface. (1) The Si NW presenting a native oxide on its surface was functionalized with APTES. (2) Glutaraldehyde was linked to the amino groups. (3) Terminal 3′-amino groups of the capture DNA strands were chemically bound to the aldehyde groups. (4) Complementary Alexa 488 fluortagged target DNA was hybridized to the capture DNA on the vSiNWs surface.

FIG. 22 shows the mean fluorescence comparison of the etched vSiNWs relative to unetched planar Si analyzed utilizing two-way ANOVA with Tukey's test indicating that the vSiNW samples exhibited six times higher fluorescence signal than planar samples, due to significantly higher NW surface area available for DNA conjugation. This increased DNA conjugation on vSiNW surfaces would allow for sensitive detection of cancer related gene mutations utilizing the vSiNW-diode biosensor platform. In addition, notably, we utilized 20 nM of capture and target DNA, which is lower than the clinically relevant cutoff concentration for BRAFV600E (11.2 μM) in CRC patients, confirming the potential clinical relevance of the biosensor platform. We also exposed the functionalized planar Si and vSiNW samples to hot water (90° C.) and measured a near zero mean fluorescence after the hot water exposure, confirming that no non-specific binding occurred. These results establish the feasibility of high sensitivity and selectivity ctDNA detection via the vSiNW biosensor platform.

### Example 4: Quantification of DNA Amount on Surface of SiNW

Where DNA is used, the DNA amount on the surface of the SiNWs may be quantified such as by using NanoDrop and a fluorescent microplate reader.

### Method

SiNWs of length 5000 nm were used. A square-shaped SiNW surface was cut, placed in scintillation vials, and exposed to UV light for 30 minutes for sterilization. Then samples were rinsed with 1 mL of acetone, 1 mL of propane-2-ol, and 3 times with NANO pure Diamon water (Barnstead). After removing the solvents, samples were incubated with 10% APTES solution (solution prepared in toluene) for 1 hr at room temperature to activate the surface with an amine-terminated moiety. Then the samples were rinsed with 1 mL of toluene, 1 mL methanol, and NANO pure Diamon water. After the rinsing process, samples were incubated with 2.5% Glutaraldehyde for 30 minutes at room temperature to link to the amino groups and present aldehyde groups on the sample's surface. Finally, the samples were rinsed with NANO pure Diamon water. Next, a solution of synthetic capture DNA was diluted with PBS buffer to provide a 10 μg/mL and used to incubate the samples for 1 hr at room temperature to ensure effective immobilization.

### Sample Withdrawal of Capture DNA at Different Time Points:

The amount of capture DNA bound to the surface was quantified using the indirect method. While the SiNW was immersed in capture DNA solution of concentration (10 μg/mL), the concentration of that solution was measured at predetermined time points (10, 20, 30, 45, 60, 90, 120 mins) using NanoDrop spectrophotometer. Then the concentration was subtracted from the initial concentration, and the amount was measured based on the solution volume and standardized per 1 mm² surface area of the SiNW.

Hybridization of Tagged Complementary Target DNA with the Capture DNA:

After incubating the samples with synthetic capture DNA for 1 hr at room temperature, the surface was rinsed with PBS. Unreacted aldehyde groups were blocked through reactions with ethanolamine for 1 hr at room temperature. Again, samples were rinsed with PBS and incubated with strand of synthetic complementary fluorescently labeled DNA; we call target DNA to hybridize to the capture DNA on the surface of SiNW. Alexa 488 unit was used as a fluorescent unit and bound to 3′ of the target DNA. The SiNW surfaces with a nanowire length of 5000 nm were incubated with a solution of the synthetic target DNA diluted with PBS buffer to provide a 10 μg/mL, 5.5 μg/mL, and 1 μg/mL for 1 hr at room temperature.

### Sample Withdrawal of Tagged Target DNA at Different Time Points:

The amount of target DNA bound to the surface was quantified using the indirect method. While the SiNW was immersed in a target DNA solution of concentrations (10, 5.5, & 1 μg/mL), the concentration of these solutions was measured at predetermined time points (10, 20, 30, 45, and 60 mins) using a fluorescent microplate reader. Then the concentration was subtracted from the initial concentration, and the amount was measured based on the solution volume and standardized per 1 mm² surface area of the SiNW. A calibration curve between the working concentrations (1-10 μg/mL) of the tagged target DNA and the fluorescent intensity was generated. The excitation wavelength of Alex488 is 490 nm and the emission wavelength is 525 nm.

### Results

### Quantity of Capture DNA Binding to the Surface:

The quantity of capture DNA was measured at different time intervals (10, 20, 30, 45, 60, 90, and 120 mins). It was noticed that the amount increased with time and reached a plateau within 45 mins, as shown in the following table.

Bound amount of capture DNA (μg) on SiNW (NW length=5000 nm) at each time point, standardized per 1 mm² surface area. N=2, data expressed as mean±SD.

| Time (mins) | 10 | 20 | 30 | 45 | 60 | 90 | 120 |
|---|---|---|---|---|---|---|---|
| Concentration | 0.006 ± 0.005 | 0.014 ± 0.002 | 0.021 ± 0.021 | 0.029 ± 0.009 | 0.028 ± 0.003 | 0.027 ± 0.003 | 0.026 ± 0.006 |
| (μg/mL) ± SD | | | | | | | |

### Calibration Curve of the Tagged Target DNA:

A calibration curve between the working concentration (1-10 μg/mL) of the tagged target DNA and the fluorescent intensity was generated. The excitation wavelength of Alex488 is 490 nm, and the emission wavelength is 525 nm. The calibration curve is given by the equation: (Fluorescent intensity (AU)=629.1 Conc. (μg/mL)+58.31). R²=0.9982.

### Quantity of Target DNA Binding to the Surface:

The quantity of tagged target DNA was measured after incubating the SiNW surface with three different concentrations of target DNA (10, 5.5, and 1 μg/mL), and the quantity was measured at different time intervals (10, 20, 30, 45, and 60 mins). It was noticed that the amount of target DNA bound on the surface increases with the increase of the initial concentration of the target DNA, and the amount increases with time at each concentration. As noticed in the following table.

Bound amount of capture DNA (μg) on SiNW (NW length=5000 nm) at different initial target DNA concentrations at each time point, standardized per 1 mm² surface area. N=1.

| Initial Target | | | | | | | |
|---|---|---|---|---|---|---|---|
| DNA | | | | | | | |
| Concentration | | Time (mins) | 10 | 20 | 30 | 45 | 60 |
| 10 | μg/mL | Concentration | 0.071 | 0.143 | 0.232 | 0.306 | 0.385 |
| | | (μg/mL) | | | | | |
| 5.5 | μg/mL | | 0.058 | 0.084 | 0.127 | 0.162 | 0.202 |
| 1 | μg/mL | | 0.005 | 0.017 | 0.025 | 0.034 | 0.043 |

### Example 5: Compare Different Lengths of NWs and Different Concentrations of Target DNA

As previously mentioned, DNA may be present on the surface of the SiNWs. It is to be understood that different lengths of SiNWs may be used and different concentrations of target DNA may be present. According to one aspect evaluation of the immobilization of DNA aptamer on the surface of different lengths of the silicon nanowire SiNWs was performed using lengths of 500 nm and 5000 nm. Evaluation of the hybridization of target DNA to an immobilized unlabeled capture DNA was performed. An evaluation of hybridization of different concentrations of tagged target DNA to an immobilized unlabeled capture DNA using SiNWs with 5000 nm length was performed.

### Experimental Materials:

Experimental materials used included organic solvents and DNA aptamers. Organic solvents included Acetone (Sigma Aldrich, St. Louis, MO), 3-aminopropyltriethoxysilane (Acros Organics, NJ), Toluene (Sigma Aldrich, St. Louis, MO), Methanol (Fisher Scientific), Propan-2-ol (Fisher Scientific, Waltham, MI), ethanolamine (Sigma Aldrich, Milwaukee, WI). DNA aptamers included: Alexa fluor 488 tagged immobilized DNA (sequence; \5′ Alex488N \-ACT GTA GCT AGA CCA AAA TCA CCT ATT-3′), capture DNA (sequence; 5′-AAT AGG TGA TTT TGG TCT AGC TAC AGT-3′) and Alexa fluor 488 tagged target DNA (sequence; 5′-ACT GTA GCT AGA CCA AAA TCA CCT ATT-\3′ Alex488N\), DNA-aptamers were obtained from (Integrated DNA, Coralville, IA).

### Self-Assembly of Capture DNA on the SiNWs Surface:

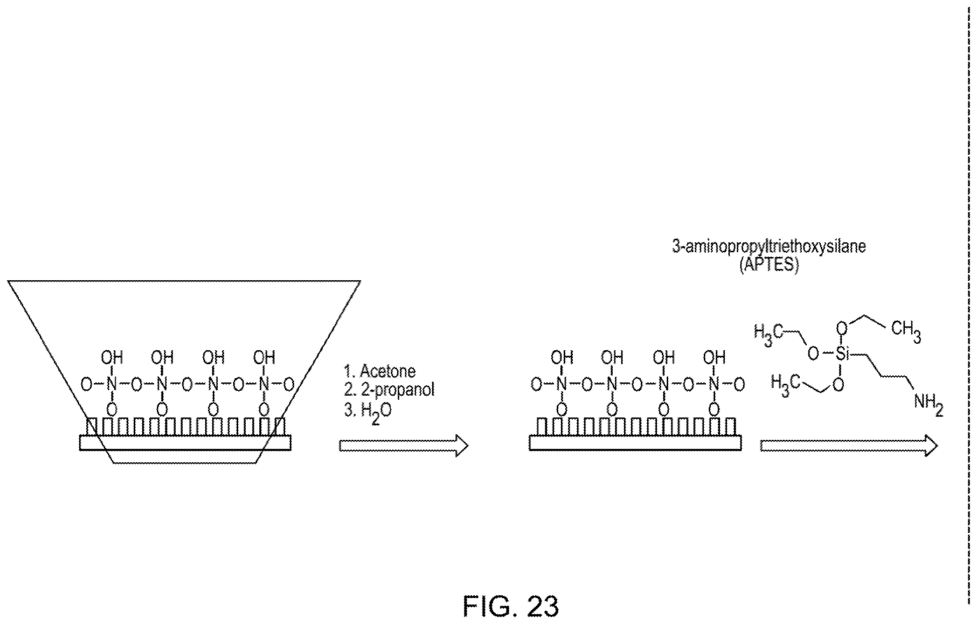

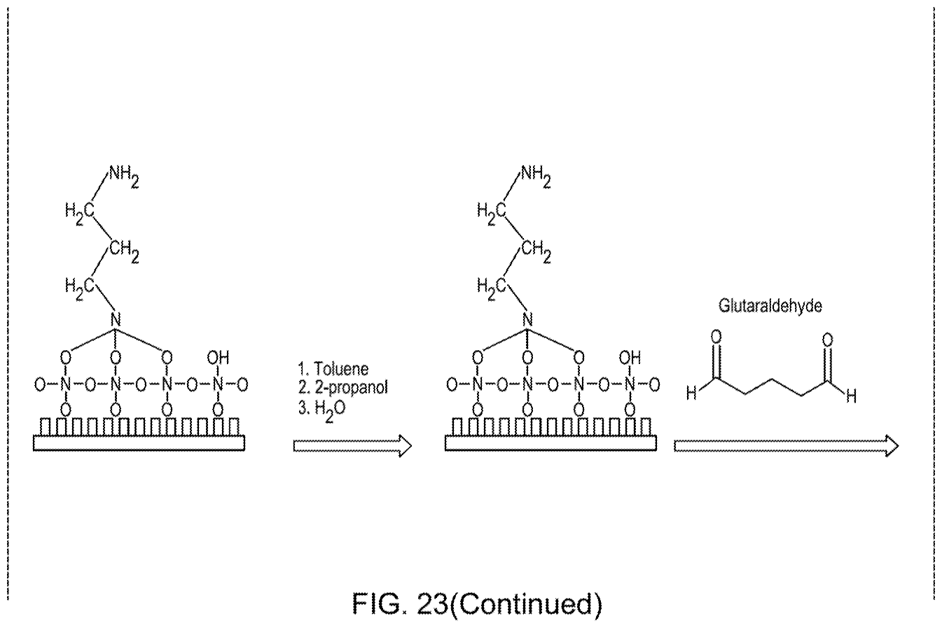

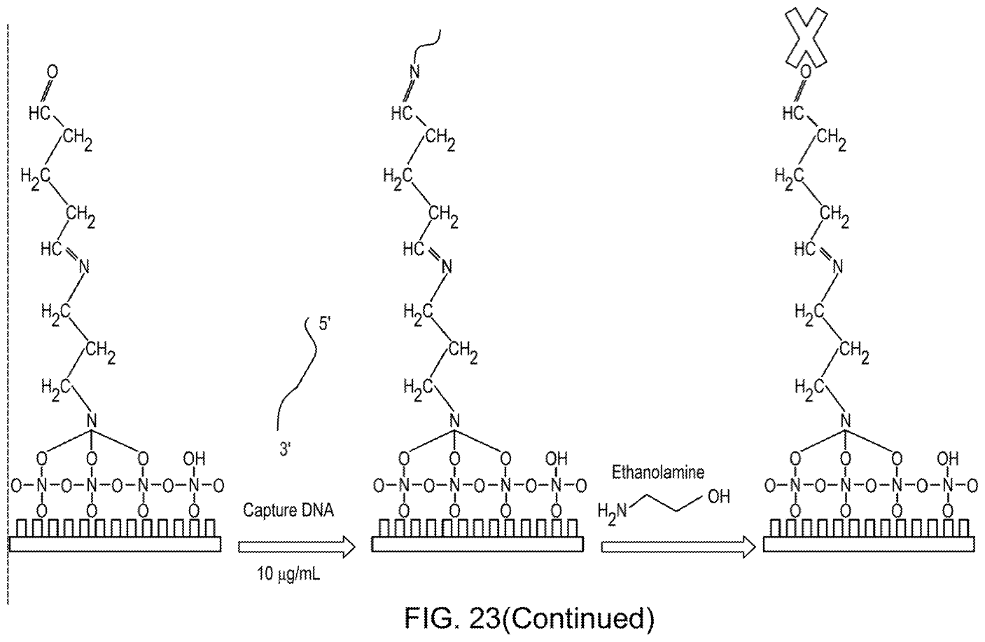

SiNWs were tested for the differences in their length, therefore two lengths of SiNWs were used, 500 nm and 5000 nm. A square shaped SiNW surface were cut and placed in scintillation vials and exposed for UV light for 30 minutes for sterilization. Then samples were rinsed with 1 mL of acetone then 1 mL of propan-2-ol, then 3 times with NANO pure Diamon water (Barnstead). After removing the solvents, samples were incubated with 10% APTES solution (solution prepared in toluene), for 1 hr at room temperature to activate the surface with an amine terminated moiety. Then the samples were rinsed with 1 mL of toluene, then 1 mL methanol and then with NANO pure Diamon water. Following the rinsing process, samples were incubated with 2.5% Glutaraldehyde for 30 minutes at room temperature to link to the amino groups to present aldehyde groups on the surface of the sample. The samples were rinsed with NANO pure Diamon water. A solution of synthetic capture DNA was diluted with PBS buffer to provide a 10 μg/mL and used to incubate the samples with for 1 hr at room temperature to ensure effective immobilization. Then the surface was rinsed with PBS, and un-reacted aldehyde groups were blocked through reactions with ethanolamine, by incubating the samples with ethanolamine for 1 hr at room temperature. Then samples were rinsed with PBS. FIG. 23 illustrates one example of such a workflow.

### Immobilization of DNA on SiNW

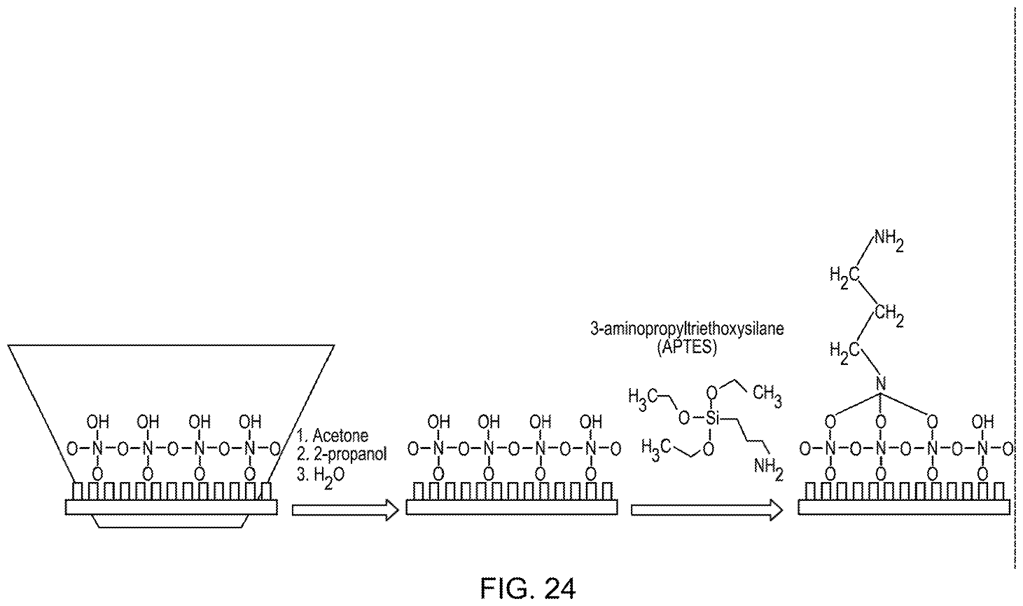

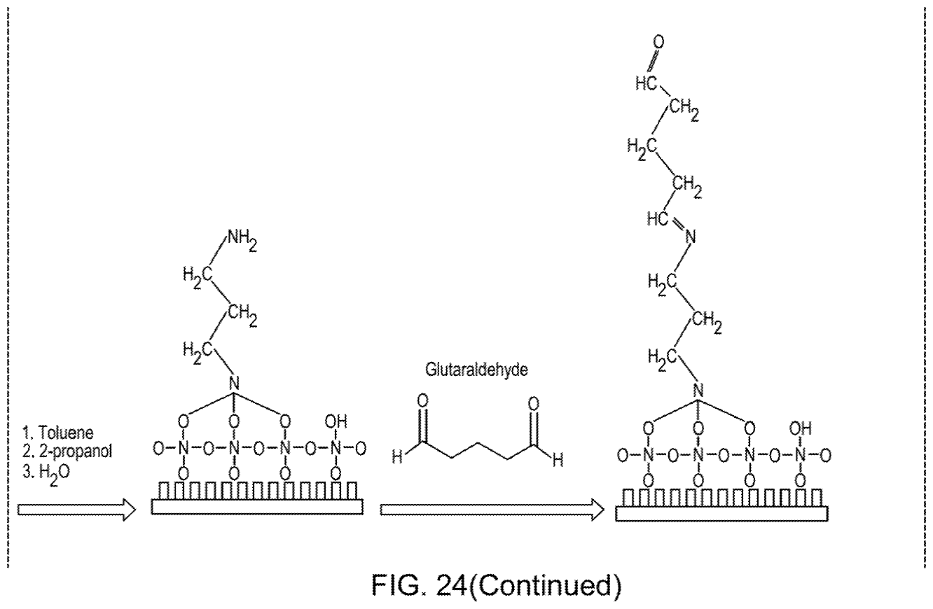

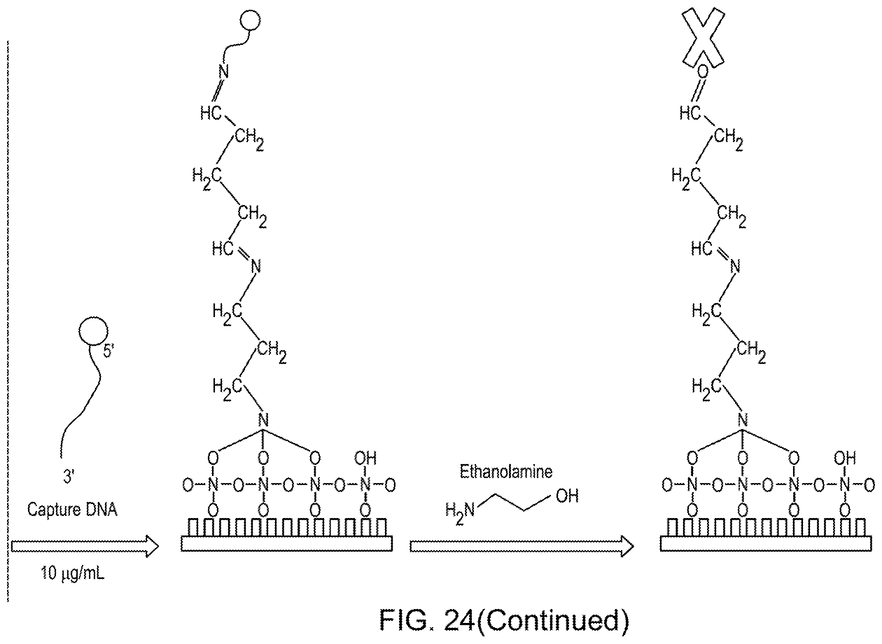

To evaluate the efficient immobilization of the DNA aptamer on the surface, a solution of a fluorescently tagged DNA sample at concentration 10 μg/mL with Alexa 488 on 5′ end was used to activate the SiNWs samples. Green fluorescence images were observed using a confocal microscope. FIG. 24 illustrates an example of such a workflow.

### Results

Fluorescent images of the SiNWs after immobilization of immobilized tagged DNA were obtained and the fluorescent intensity has been quantified and found to be 36463±6457 and 6059±425 in the SiNWs with length 500 nm and 5000 nm, respectively. To detect BRAFV600E mutation gene, we immobilized a 27-mer capture DNA strand, onto the surface. This capture DNA sequence is fully complementary to that of the target DNA which is partial sequence of the BRAFV600E mutation gene. Fluorescence imagery confirms the successful hybridization of the 10 μg/ml Alexa488-labeled target DNA to the capture DNA, which reveals the ability of the SiNWs to detect cancer-related mutation genes in both SiNWs length that have been used. The fluorescent intensity has been quantified using image J, the fluorescent intensity of the labeled target DNA hybridized with immobilized unlabeled capture DNA Is 18200±5265 and 1571±36.52 in the SiNWs with length 500 nm and 5000 nm, respectively. It has been found that the fluorescent intensity of the hybridized target DNA in the presence of capture DNA using a length of 5000 nm is significantly higher than that using a length of 500 nm. This difference is attributed to the fact that the longer SiNWs has higher surface area than that of the shorter SiNWs. For the next step we decided to use the SiNWs of 5000 nm length and check how the concentration of the target DNA would affect the fluorescent intensity.

Fluorescent images were obtained for the SiNWs after hybridization with different concentrations of tagged target DNA in the presence of capture DNA using a length of 5000 nm. The concentration work of the target DNA was 10 μg/mL, 7.5 μg/mL, 3.5 μg/mL, 2 μg/mL, and 1 μg/mL. The fluorescent intensity has been quantified and was 18200±5265, 13392±2608, 6505±916.6, 4427±986.9, and 2703±592.9 for the concentrations 10 μg/mL, 7.5 μg/mL, 5.5 μ/ml, and 3.5 μg/mL, respectively.

Thus, it should be understood that different lengths of SiNWs are contemplated, different concentrations of tagged target DNA are contemplated in some embodiments.

### Options, Variations, and Alternatives

Although various embodiments have been shown and described including those set forth in the appendices, it is to be understood that the present invention contemplates numerous options, variations, and alternatives. These include the manner in which the biosensor is functionalized for detection of different analytes, the number of biosensors operating in parallel, the manufacturing methods used, the type of processor used, the type of user interface, the manner in which results are displayed or otherwise communicated to a user. These may further include the density of the nanowires, the length of the nanowires, the selectivity of the sensors, the sensitivity of the sensors, and other variations.

It should also be understood that various structural components shown and described herein can function together with additional structural components in order to provide a biosensor and platform with improved features. For example, one of the benefits and advantages of the biosensor shown and described is a significant reduction in noise in comparison to prior art attempts for vertically-oriented silicon nanowire array-based bioelectronic sensors. Having the sensor which does not require exposure to light or use of lamps, provides for improved signal-to-noise ratios. Having very small probes further reduces noise. Using Cu tape provides a conductor with a relatively large surface area and little resistance. In addition, having an emitter doping density in a range of around 7×10¹⁷ to 1×10¹⁹ cm⁻³ and preferably in a range of around 7×10¹⁷ to 1×10¹⁸ cm⁻³ is desirable for high signal-to-noise ratio of the sensor current change. It should further be understood that the junction depth and doping densities results in a specific sheet resistance (Rₛₕₑₑₜ) in Ω/sq. One junction depth which has been used is around 0.6 μm although other junction depths may be used. Changing the doping densities and junction depth results in a specific sheet resistance. Thus, doping levels are directly related to sheet resistivity, where a high Rₛₕₑₑₜ value corresponds to a low doping concentration, and vice versa. The resulting sheet resistance also affects the current signal and so these and other parameters which may be optimized to improve sensitivity or maintain a desired sensitivity of the biosensor. Thus, these and/or other structural features allow for a biosensor to be constructed which allow small current signals to be measured with a sufficiently low amount of noise that an effective platform may be constructed such as a reasonably low cost platform capable of use within the field for testing for analytes.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment or example.

The invention is not to be limited to the particular embodiments described herein. In particular, the invention contemplates numerous variations in the specific methodology used and structures provided as described herein. The foregoing description has been presented for purposes of illustration and description. It is not intended to be an exhaustive list or limit any of the invention to the precise forms disclosed. It is contemplated that other alternatives or exemplary aspects are considered included in the invention. The description is merely examples of embodiments, processes, or methods of the invention. It is understood that any other modifications, substitutions, and/or additions can be made, which are within the intended spirit and scope of the invention.

### REFERENCES

[1] G. Guglielmi, The explosion of new coronavirus tests that could help to end the pandemic, Nature 583 (7817) (2020) 506-509.
[2] P. Pokhrel, C. P. Hu, H. B. Mao, Detecting the Coronavirus (COVID-19), ACS Sensors 5 (8) (2020) 2283-2296.
[3] S. A. Bustin, R. Mueller, Real-time reverse transcription PCR (qRT-PCR) and its potential use in clinical diagnosis, Clin Sci 109 (4) (2005) 365-379.
[4] I. Arevalo-Rodriguez, D. Buitrago-Garcia, D. Simancas-Racines, P. Zambrano-Achig, R. del Campo, A. Ciapponi, O. Sued, L. Martinez-Garcia, A. Rutjes, N. Low, P. M. Bossuyt, J. A. Perez-Molina, J. Zamora, False-negative results of initial RT-PCR assays for COVID-19: A systematic review, medRxiv (2020) 2020.04.16.20066787.
[5] S. Woloshin, N. Patel, A. S. Kesselheim, False Negative Tests for SARS-CoV-2 Infection-Challenges and Implications, N Engl J Med 383 (6) (2020) e38.
[6] J. Watson, P. F. Whiting, J. E. Brush, Interpreting a covid-19 test result, BMJ (2020).
[7] L. M. Kucirka, S. A. Lauer, O. Laeyendecker, D. Boon, J. Lessler, Variation in False-Negative Rate of Reverse Transcriptase Polymerase Chain Reaction-Based SARS-CoV-2 Tests by Time Since Exposure, Annals of Internal Medicine 173 (4) (2020) 262-267.
[8] Interim Guidance for Antigen Testing for SARS-CoV-2, 2022. https://www.cdc.gov/coronavirus/2019-ncov/lab/resources/antigen-tests-guidelines.html #table1.
[9] N. P. Ambulos, Jr., The Good, the Bad and the Ugly: Getting CLIA-Certification for a Basic Research Lab, J Biomol Tech 24 (Suppl) (2013) S21-S21.
[10] Antigen-detection in the diagnosis of SARS-CoV-2 infection (https://www.who.int/publications/i/item/antigen-detection-in-the-diagnosis-of-sars-cov-2infection-using-rapid-immunoassays), in: W. H. Organization (Ed.) 2021.
[11] R. W. Peeling, P. L. Olliaro, D. I. Boeras, N. Fongwen, Scaling up COVID-19 rapid antigen tests: promises and challenges, The Lancet Infectious Diseases 21 (9) (2021) e290-e295.
[12] Y. Yang, Y. Peng, C. Lin, L. Long, J. Hu, J. He, H. Zeng, Z. Huang, Z.-Y. Li, M. Tanemura, J. Shi, J. R. Lombardi, X. Luo, Human ACE2-Functionalized Gold “Virus-Trap” Nanostructures for Accurate Capture of SARS-CoV-2 and Single-Virus SERS Detection, Nano-Micro Letters 13 (1) (2021) 109.
[13] S. Park, H. Kim, K. Woo, J.-M. Kim, H.-J. Jo, Y. Jeong, K. H. Lee, SARS-CoV-2 Variant Screening Using a Virus-Receptor-Based Electrical Biosensor, Nano Letters 22 (1) (2022) 50-57.
[14] J.-H. Lee, M. Choi, Y. Jung, S. K. Lee, C.-S. Lee, J. Kim, J. Kim, N. H. Kim, B.-T. Kim, H. G. Kim, A novel rapid detection for SARS-CoV-2 spike 1 antigens using human angiotensin converting enzyme 2 (ACE2), Biosensors and Bioelectronics 171 (2021) 112715.
[15] R. L. Pinals, F. Ledesma, D. Yang, N. Navarro, S. Jeong, J. E. Pak, L. Kuo, Y.-C. Chuang, Y.-W. Cheng, H.-Y. Sun, M. P. Landry, Rapid SARS-CoV-2 Spike Protein Detection by Carbon Nanotube-Based Near-Infrared Nanosensors, Nano Letters 21 (5) (2021) 2272-2280.
[16] E. D. Nascimento, W. T. Fonseca, T. R. de Oliveira, C. R. S. T. B. de Correia, V. M. Faça, B. P. de Morais, V. C. Silvestrini, H. Pott-Junior, F. R. Teixeira, R. C. Faria, COVID-19 diagnosis by SARS-CoV-2 Spike protein detection in saliva using an ultrasensitive magneto-assay based on disposable electrochemical sensor, Sensors and Actuators B: Chemical 353 (2022) 131128.
[17] J. Yang, S. L. Petitjean, M. Koehler, Q. Zhang, A. C. Dumitru, W. Chen, S. Derclaye, S. P. Vincent, P. Soumillion, D. Alsteens, Molecular interaction and inhibition of SARS-CoV-2 binding to the ACE2 receptor, Nature Communications 11 (1) (2020) 4541.
[18] S. Ozono, Y. Zhang, H. Ode, K. Sano, T. S. Tan, K. Imai, K. Miyoshi, S. Kishigami, T. Ueno, Y. Iwatani, T. Suzuki, K. Tokunaga, SARS-CoV-2 D614G spike mutation increases entry efficiency with enhanced ACE2-binding affinity, Nature Communications 12 (1) (2021) 848.
[19] K. Kim, C. Park, D. Kwon, D. Kim, M. Meyyappan, S. Jeon, J. S. Lee, Silicon nanowire biosensors for detection of cardiac troponin I (cTnI) with high sensitivity, Biosens Bioelectron 77 (2016) 695-701.
[20] G.-J. Zhang, Y. Ning, Silicon nanowire biosensor and its applications in disease diagnostics: A review, Analytica Chimica Acta 749 (2012) 1-15.
[21] R. Smith, W. Duan, J. Quarterman, A. Morris, C. Collie, M. Black, F. Toor, A. K. Salem, Surface Modifying Doped Silicon Nanowire Based Solar Cells for Applications in Biosensing, Advanced Materials Technologies 4 (2) (2019) 1800349.
[22] G. H. Yu, C. M. Lieber, Assembly and integration of semiconductor nanowires for functional nanosystems, Pure Appl Chem 82 (12) (2010) 2295-2314.
[23] F. Patolsky, G. F. Zheng, C. M. Lieber, Fabrication of silicon nanowire devices for ultrasensitive, label-free, real-time detection of biological and chemical species, Nat Protoc 1 (4) (2006) 1711-1724.
[24] A. I. Hochbaum, R. Fan, R. R. He, P. D. Yang, Controlled growth of Si nanowire arrays for device integration, Nano Letters 5 (3) (2005) 457-460.
[25] R. S. Wagner, W. C. Ellis, Vapor-Liquid-Solid Mechanism of Single Crystal Growth (New Method Growth Catalysis from Impurity Whisker Epitaxial+Large Crystals Si E), Appl Phys Lett 4 (5) (1964) 89-&.
[26] F. Toor, J. B. Miller, L. M. Davidson, W. Q. Duan, M. P. Jura, J. Yim, J. Forziati, M. R. Black, Metal assisted catalyzed etched (MACE) black Si: optics and device physics, Nanoscale 8 (34) (2016) 15448-15466.
[27] F. Toor, J. B. Miller, L. M. Davidson, L. Nichols, W. Q. Duan, M. P. Jura, J. Yim, J. Forziati, M. R. Black, Nanostructured silicon via metal assisted catalyzed etch (MACE): chemistry fundamentals and pattern engineering, Nanotechnology 27 (41) (2016).
[28] J. Shang, G. Ye, K. Shi, Y. Wan, C. Luo, H. Aihara, Q. Geng, A. Auerbach, F. Li, Structural basis of receptor recognition by SARS-CoV-2, Nature (2020).
[29] Y. Huang, C. Yang, X. F. Xu, W. Xu, S. W. Liu, Structural and functional properties of SARS-CoV-2 spike protein: potential antivirus drug development for COVID-19, Acta Pharmacol Sin 41 (9) (2020) 1141-1149.
[30] P. R. Nair, M. A. Alam, Design considerations of silicon nanowire biosensors, IEEE T Electron Dev 54 (12) (2007) 3400-3408.
[31] H. Kai, M. Kai, Interactions of coronaviruses with ACE2, angiotensin II, and RAS inhibitors-lessons from available evidence and insights into COVID-19, Hypertens Res (2020).
[32] D. S. Waugh, An overview of enzymatic reagents for the removal of affinity tags, Protein expression and purification 80 (2) (2011) 283-93.
[33] J. Johnson, Y. Zhai, H. Salimi, N. Espy, N. Eichelberger, O. DeLeon, Y. O'Malley, J. Courter, A. B. Smith, 3rd, N. Madani, J. Sodroski, H. Haim, Induction of a Tier-1-Like Phenotype in Diverse Tier-2 Isolates by Agents That Guide HIV-1 Env to Perturbation-Sensitive, Nonnative States, J Virol 91 (15) (2017).
[34] O. DeLeon, H. Hodis, Y. O'Malley, J. Johnson, H. Salimi, Y. Zhai, E. Winter, C. Remec, N. Eichelberger, B. Van Cleave, R. Puliadi, R. D. Harrington, J. T. Stapleton, H. Haim, Accurate predictions of population-level changes in sequence and structural properties of HIV-1 Env using a volatility-controlled diffusion model, PLOS biology 15 (4) (2017) e2001549.
[35] R. Wolfel, V. M. Corman, W. Guggemos, M. Seilmaier, S. Zange, M. A. Muller, D. Niemeyer, T. C. Jones, P. Vollmar, C. Rothe, M. Hoelscher, T. Bleicker, S. Brunink, J. Schneider, R. Ehmann, K. Zwirglmaier, C. Drosten, C. Wendtner, Virological assessment of hospitalized patients with COVID-2019, Nature 581 (7809) (2020) 465-469.
[36] Z. Ke, J. Oton, K. Qu, M. Cortese, V. Zila, L. McKeane, T. Nakane, J. Zivanov, C. J. Neufeldt, J. M. Lu, Julia Peukes, Xiaoli Xiong, H. G. Kräusslich, S. H. W. Scheres, R. Bartenschlager, J. A. G. Briggs, Structures, conformations and distributions of SARS-CoV-2 spike protein trimers on intact virions, bioRxiv (2020).
[37] J. S. Mandeville, H. A. Tajmir-Riahi, Complexes of Dendrimers with Bovine Serum Albumin, Biomacromolecules 11 (2) (2010) 465-472.
[38] N. Lloret, R. S. Frederiksen, T. C. Møller, N. I. Rieben, S. Upadhyay, L. De Vico, J. H. Jensen, J. Nygård, K. L. Martinez, Effects of buffer composition and dilution on nanowire field-effect biosensors, Nanotechnology 24 (3) (2013).
[39] F. Krebs, C. Scheller, K. Grove-Heike, L. Pohl, H. Wätzig, Isoelectric point determination by imaged CIEF of commercially available SARS-CoV-2 proteins and the hACE2 receptor, Electrophoresis 42 (6) (2021) 687-692.
[40] W. Duan, B. Gao, K. A. S. M. E. Haque, F. Toor, Performance enhancement techniques for the front and back of nanostructured “black silicon” solar cells, Journal of Photonics for Energy 8 (3) (2018) 034001.
[41] W. Shockley, The theory of p-n junctions in semiconductors and p-n junction transistors, The Bell System Technical Journal 28 (3) (1949) 435-489.
[42] F. Hernando., R. Gutierrez., G. Bueno., F. Recart., V. Rodriguez., Humps, a surface damage explanation, 2nd World Conference and Exhibition on Photovoltaic Solar Energy Conversion, Vienna, Austria, 1998.
[43] K. R. McIntosh, Lumps, humps and bumps: three detrimental effects in the current-voltage curve of silicon solar cells, 2001.
[44] K. R. McIntosh, L. E. Black, On effective surface recombination parameters, Journal of Applied Physics 116 (1) (2014).
[45] S. M. Sze, K. K. Ng, Physics of semiconductor devices, Third edition ed., Wiley-Interscience 2007.
[46] P. R. Nair, M. A. Alam, Screening-Limited Response of NanoBiosensors, Nano Letters 8 (5) (2008) 1281-1285.
[47] Y. M. Loo, P. M. Mc Tamney, R. H. Arends, M. E. Abram, A. A. Aksyuk, S. Diallo, D. J. Flores, E. J. Kelly, K. Ren, R. Roque, K. Rosenthal, K. Streicher, K. M. Tuffy, N. J. Bond, O. Cornwell, J. Bouquet, L. I. Cheng, J. Dunyak, Y. Huang, A. I. Rosenbaum, V. P. Reddy, H. Andersen, R. H. Carnahan, J. E. Crowe, Jr., A. I. Kuehne, A. S. Herbert, J. M. Dye, H. Bright, N. L. Kallewaard, M. N. Pangalos, M. T. Esser, The SARS-CoV-2 monoclonal antibody combination, AZD7442, is protective in non-human primates and has an extended half-life in humans, Sci Transl Med (2022) eab18124
[48] Upadhyaya, A.; Sheoran, M.; Rohatgi, A., High-efficiency screen-printed belt co-fired solar cells on cast multicrystalline silicon. Appl Phys Lett 2005, 86 (5).
[49] Zagozdzonwosik, W.; Grabiec, P. B.; Lux, G., Silicon Doping from Phosphorus Spin-on Dopant Sources in Proximity Rapid Thermal-Diffusion. J Appl Phys 1994, 75 (1), 337-344.
[50] Zhu, M.; Lerum, M. Z.; Chen, W., How to prepare reproducible, homogeneous, and hydrolytically stable aminosilane-derived layers on silica. Langmuir 2012, 28 (1), 416-423.
[51] 2400 SourceMeter® Line Datasheet. https://www.testequipmenthq.com/datasheets/KEITHLEY-2400-Datasheet.pdf.
[52] Shockley, W., The theory of p-n junctions in semiconductors and p-n junction transistors. The Bell System Technical Journal 1949, 28 (3), 435-489.
[53] Sze, S. M.; Ng, K. K., Physics of semiconductor devices. Third edition ed.; Wiley-Interscience: 2007; p 815 pages.
[54] McIntosh, K. R., Lumps, humps and bumps: three detrimental effects in the current-voltage curve of silicon solar cells. 2001.
[55] Hernando., F.; Gutierrez., R.; Bueno., G.; Recart., F.; Rodriguez., V. In Humps, a surface damage explanation, 2nd World Conference and Exhibition on Photovoltaic Solar Energy Conversion, Vienna, Austria, Vienna, Austria, 1998.
[56] Hallam, B. J.; Hamer, P. G.; Bonilla, R. S.; Wenham, S. R.; Wilshaw, P. R., Method of Extracting Solar Cell Parameters From Derivatives of Dark I-V Curves. Ieee J Photovolt 2017, 7 (5), 1304-1312.
[57] Tian, B. Z.; Cohen-Karni, T.; Qing, Q.; Duan, X. J.; Xie, P.; Lieber, C. M., Three-Dimensional, Flexible Nanoscale Field-Effect Transistors as Localized Bioprobes. Science 2010, 329 (5993), 830-834.
[58] Qing, Q.; Pal, S. K.; Tian, B. Z.; Duan, X. J.; Timko, B. P.; Cohen-Karni, T.; Murthy, V. N.; Lieber, C. M., Nanowire transistor arrays for mapping neural circuits in acute brain slices. P Natl Acad Sci USA 2010, 107 (5), 1882-1887.
[59] Jiang, Z.; Qing, Q.; Xie, P.; Gao, R. X.; Lieber, C. M., Kinked p-n Junction Nanowire Probes for High Spatial Resolution Sensing and Intracellular Recording. Nano Lett 2012, 12 (3), 1711-1716.
[60] Nair, P. R.; Alam, M. A., Design considerations of silicon nanowire biosensors. Ieee T Electron Dev 2007, 54 (12), 3400-3408.
[61] Cheng, M.; Cuda, G.; Bunimovich, Y.; Gaspari, M.; Heath, J.; Hill, H.; Mirkin, C.; Nijdam, A.; Terracciano, R.; Thundat, T., Nanotechnologies for biomolecular detection and medical diagnostics. Current Opinion in Chemical Biology 2006, 10 (1), 11-19.
[62] Zheng, G.; Patolsky, F.; Cui, Y.; Wang, W. U.; Lieber, C. M., Multiplexed electrical detection of cancer markers with nanowire sensor arrays. Nat Biotechnol 2005, 23 (10), 1294-1301.

What is claimed is:

1. A vertically-oriented silicon nanowire-array based bioelectronic sensor platform comprising:
a vertically-oriented silicon nanowire-array bioelectronic sensor comprising:
a silicon substrate doped to function as an electrically active p-n junction diode with a p-doped base and an n+-doped emitter and having a vertically-oriented silicon nanowire-array at the n+-doped emitter;
a conductive contact positioned at the silicon substrate and in electrical connection with the vertically-oriented silicon nanowire-array;
a dielectric stack overlaying the conductive contact; and
a back contact associated with a back surface of the silicon substrate.

2. The vertically-oriented silicon nanowire-array based bioelectronic sensor platform of claim 1 further comprising a housing comprising a top portion and a bottom portion and configured such that the top portion has an opening aligned with a sensing area comprising the vertically-oriented silicon nanowire-array of the vertically-oriented silicon nanowire-array bioelectronic sensor.

3. The vertically-oriented silicon nanowire-based bioelectronic sensor platform of claim 2 further comprising a first conductor electrically connected to the conductive contact and a second conductor electrically connected to the back contact to provide first and second terminals for the vertically-oriented silicon nanowire-array bioelectronic sensor.

4. The vertically-oriented silicon nanowire-based bioelectronic sensor platform of claim 3 wherein the first conductor and the second conductor are both conductive tape.

5. The vertically-oriented silicon nanowire-based bioelectronic sensor platform of claim 1 further comprising a layer of insulative tape between the vertically-oriented silicon nanowire-array bioelectronic sensor and the top portion of the housing.

6. The vertically-oriented silicon nanowire-based bioelectronic sensor platform of claim 1 further comprising a current sensor electrically connected to the vertically-oriented silicon nanowire-based bioelectronic sensor.

7. The vertically-oriented silicon nanowire-based bioelectronic sensor platform of claim 6 further comprising a processor electrically connected to the current sensor.

8. The vertically-oriented silicon nanowire-based bioelectronic sensor platform of claim 7 further comprising a user interface device operatively connected to the processor.

9. The vertically-oriented silicon nanowire-based bioelectronic sensor platform of claim 8 wherein the user interface device is a touchscreen display.

10. The vertically-oriented silicon nanowire-based bioelectronic sensor platform of claim 1 wherein the vertically-oriented silicon nanowire-based bioelectronic sensor is functionalized for detection of analytes selected from a set consisting of cancer cell antigens, ctDNA mutations, coronavirus spike protein, and a hormone.

11. The vertically-oriented silicon nanowire array-based bioelectronic sensor platform of claim 1 wherein the vertically-oriented silicon nanowire-array comprises etched vertical silicon nanowires of at least about 350 nm in length and at least about 10¹⁰ per 1 cm² in density

12. The vertically-oriented silicon nanowire array-based bioelectronic sensor platform of claim 1 wherein the vertically-oriented silicon nanowire-array bioelectronic sensor is a first sensor functionalized for a first analyte and wherein the vertically-oriented silicon nanowire array-based bioelectronic sensor platform further comprises a second sensor functionalized for a second analyte, the first analyte different from the second analyte.

13. The vertically-oriented silicon nanowire array-based bioelectronic sensor platform of claim 1 wherein the vertically-oriented silicon nanowire array-based bioelectronic sensor is fabricated using a metal-assisted chemical etching (MACE) process.

14. The vertically-oriented silicon nanowire array-based bioelectronic sensor platform of claim 1 wherein the vertically-oriented silicon nanowire array-based bioelectronic sensor has n+-doping in a range of 7×10¹⁷ to 1×10¹⁹ cm⁻³.

15. A method comprising biofunctionalizing the vertically-oriented silicon nanowire array-based bioelectronic sensor of the vertically-oriented silicon nanowire-array based bioelectronic sensor platform claim 1.

16. A method comprising testing for an analyte using the vertically-oriented silicon nanowire-array based bioelectronic sensor platform of claim 1.

17. A vertically-oriented silicon nanowire array-based bioelectronic sensor platform comprising:
a first vertical silicon nanowire array-based biosensor;
a second vertical silicon nanowire array-based biosensor in parallel with the first vertical silicon nanowire array-based biosensor;
a current sensor electrically connected to the first vertical silicon nanowire array-based biosensor and the second vertical silicon nanowire array-based biosensor to provide for multiplexed detection of a first analyte with the first vertical silicon nanowire-array based biosensor and a second analyte with the second vertical silicon nanowire array-based biosensor;
wherein each of the first vertical nanowire array-based biosensor and the second vertical silicon nanowire array-based biosensor comprises a silicon substrate doped to function as an electrically active p-n junction diode with a p-doped base and an n+-doped emitter and having a vertically-oriented silicon nanowire-array at the n+-doped emitter, a conductive contact positioned at the silicon substrate and in electrical connection with the vertically-oriented silicon nanowire-array, a dielectric stack overlaying the conductive contact, and a back contact associated with the back surface of the silicon substrate;
wherein the first vertical silicon nanowire array-based biosensor is functionalized for detecting the first analyte;
wherein the second vertical silicon nanowire array-based biosensor is functionalized for detecting the second analyte.

18. The vertically-oriented silicon nanowire array-based bioelectronic sensor platform of claim 17 further comprising a processor electrically connected to the current sensor.

19. The vertically-oriented silicon nanowire array-based bioelectronic sensor platform of claim 18 further comprising a user interface device operatively connected to the processor.

20. The vertically-oriented silicon nanowire array-based bioelectronic sensor platform of claim 19 wherein the user interface device is a touchscreen display.

21. The vertically-oriented silicon nanowire array-based bioelectronic sensor platform of claim 17 wherein the first analyte is a cancer cell antigen.

22. The vertically-oriented silicon nanowire array-based bioelectronic sensor platform of claim 21 wherein the second analyte is a cancer cell antigen, the first analyte different from the second analyte.

23. The vertically-oriented silicon nanowire array-based bioelectronic sensor platform of claim 17 wherein the first analyte is selected from a set consisting of a ctDNA mutation, a coronavirus spike protein, a hormone, an estrogenic compound.

24. The vertically-oriented silicon nanowire array-based bioelectronic sensor platform of claim 17 wherein the first vertical silicon nanowire array-based biosensor comprises etched vertical silicon nanowire arrays of at least about 350 nm in length and at least about 10¹⁰ per 1 cm² in density and wherein the second vertical silicon nanowire array-based biosensor comprises etched vertical silicon nanowire arrays of at least about 350 nm in length and at least about 10¹⁰ per 1 cm².

25. The vertically-oriented silicon nanowire array-based bioelectronic sensor platform of claim 17 wherein the first vertical silicon nanowire array-based biosensor and the second vertical nanowire array-based biosensor are fabricated using a metal-assisted chemical etching (MACE) process.

26. The vertically-oriented silicon nanowire array-based bioelectronic sensor platform of claim 15 wherein sheet resistivity is selected to increase sensitivity of sensor response.

27. A method comprising:
fabricating a vertically-oriented silicon nanowire array-based bioelectronic sensor comprising a silicon substrate doped to function as an electrically active p-n junction diode with a p-doped base and an n+-doped emitter and having a vertically-oriented silicon nanowire-array at the n+-doped emitter, a conductive contact positioned at the silicon substrate and in electrical connection with the vertically-oriented silicon nanowire-array, a dielectric stack overlaying the conductive contact, and a back contact at a back surface of the silicon substrate;
biofunctionalizing the vertically-oriented silicon nanowire array-based bioelectronic sensor.

28. The method of claim 27 further comprising calibrating I-V curves for the vertically-oriented silicon nanowire array-based bioelectronic sensor.

29. The method of claim 27 further comprising selecting a sheet resistivity to increase sensitivity of the vertically-oriented silicon nanowire array-based bioelectronic sensor and wherein the vertically-oriented silicon nanowire array-based bioelectronic sensor is fabricated to provide the sheet resistivity.

30. The method of claim 29 wherein the sheet resistivity is at least 500 Ω/sq.

31. The method of claim 29 wherein the sheet resistivity is at least 1000 Ω/sq.

32. The method of claim 29 wherein the sheet resistivity is at least 1200 Ω/sq.

33. The method of claim 29 wherein the sheet resistivity is in the range of 1000 to 1200 Ω/sq.


SEQUENCE LISTING (3 sequences)

SEQ ID NO: 1 - DNA, 27 bp, synthetic construct
  source 1..27: mol_type = other DNA
actgtagctagaccaaaatcacctatt

SEQ ID NO: 2 - DNA, 27 bp, synthetic construct
  source 1..27: mol_type = other DNA
aataggtgattttggtctagctacagt

SEQ ID NO: 3 - DNA, 27 bp, Homo sapiens
  source 1..27: mol_type = genomic DNA
actgtagctagaccaaaatcacctatt